(19) United States
(12) Patent Application Publication
(10) Pub. No.: US 2026/0167712 A1
(43) Pub. Date: Jun. 18, 2026

(54) INHIBITORY ANTIBODIES AGAINST GLUT1

(71) Applicant: ICOSAGEN CELL FACTORY OÜ, Ossu, Ülenurme, Tartumaa (EE)

(72) Inventors: Maiken ABEL, Tartu (EE); Korneelia ANTON, Tartu (EE); Denis BELITŠKIN, Tartu (EE); Paule HERMET, Tartu (EE); Erkki JURONEN, Tartu (EE); Gaily KIVI, Tartu (EE); Julia KOSKAR, Tartu (EE); Oliver MEIKAR, Tartu (EE); Andres MÄNNIK, Tartu (EE); Anu PLANKEN, Tartu (EE); Jaan-Eerik PLANKEN, Tartu (EE); Eve SANKOVSKI, Tartu (EE); Siret TAHK, Tartu (EE); Joan TEYRA, Tartu (EE); Mart USTAV, Tartu (EE); Mart USTAV Jr, Tartu (EE); Kai VIRUMÄE, Tartu (EE); Robin PAU, Tartu (EE)

(21) Appl. No.: 19/126,312

(22) PCT Filed: Nov. 1, 2023
(86) PCT No.: PCT/IB2023/061001

§ 371 Date: May 1, 2025

Related U.S. Application Data
(60) Provisional application No. 63/381,801, filed on Nov. 1, 2022.

(51) Int. Cl. C07K 16/28; A61K 31/155; A61K 31/454; A61K 39/00; A61P 3/10; A61P 35/00; G01N 33/575

(52) U.S. Cl. CPC C07K 16/28; A61K 31/155; A61K 31/454; A61P 3/10; A61P 35/00; G01N 33/575; A61K 2039/505; C07K 2317/565; C07K 2317/92; G01N 2333/705

(57) ABSTRACT

Anti SLC2A1 antibodies and antigen binding fragments thereof are disclosed. The antibodies and the fragments are suitable for use in inhibiting tumor cell glucose uptake and for reprogramming tumor cell metabolism toward oxidative phosphorylation. The disclosed antibodies and antigen binding fragments thereof are suitable for use for cancer therapy alone or in combination with compounds inhibiting oxidative phosphorylation pathway.

### SEQUENCE LISTING

This application contains a sequence listing which is submitted as a computer readable format.

### FIELD OF THE INVENTION

The present invention is related to anti-SLC2A1 monoclonal antibodies, to the use of said compounds as single agents or in combinations with pharmaceutical agents for the treatment or prophylaxis of diseases such as cancer, autoimmune diseases, inflammation, infectious diseases, and metabolic diseases, as well as to use of said compounds as intermediates for generation of new modified anti-SLC2A1 monoclonal antibodies.

### BACKGROUND OF THE INVENTION

Glucose is an essential substrate for mammalian metabolism. Transport of glucose through cell membranes requires specific transport proteins that belong to glucose transporter family. Within the family, proteins are named with symbol GLUT and genes with symbol SLC2 (Solute Carrier Family 2).

All known GLUT proteins contain 12 transmembrane domains forming a large hydrophilic cavity at the center of the protein. Amino acids located inside the cavity facilitate ligand binding and determine the substrate specificity of the transporter¹. The ligand transport is facilitated by a cycle of the conformational changes starting from ligand uptake in the outward facing (open to the extracellular medium) conformation, transition to the closed states with the ligand located in the protein cavity and the inward facing (open to cytoplasm) state allowing the ligand release^{2,3}.

GLUT family proteins are subdivided into three classes based on protein sequence and similarity. Class I is the “glucose transporters” including GLUT1, GLUT2, GLUT3, GLUT4, and GLUT14. Class II glucose transporters include GLUT5, GLUT7, GLUT9, and GLUT11, and are known to transport fructose as well. Class III is made up of GLUT6, GLUT8, GLUT10, GLUT12, and GLUT13 (HMIT1) with largely unknown substrates⁴.

Glucose is central to energy consumption in the human body. The expression and subcellular location of the most abundant mammalian glucose transporters GLUT1, GLUT2, GLUT3, GLUT4 on a particular cell type is an important aspect in glucose consumption regulation. GLUT2, GLUT3, GLUT4 are involved with specific central processes of the human body. GLUT2 is important regulator of glucose sensing and insulin secretion in pancreas and GLUT3 is mediating neuronal glucose uptake whereas GLUT4 is involved in glucose metabolism in muscle and fat cells⁵⁻⁷. GLUT1 on the other hand is regarded as a major glucose transporter during the embryonic development⁸. In adult tissues, GLUT1 is expressed on red blood cells, endothelial cells of blood-brain barrier and brown adipose tissue⁹⁻¹¹.

Importantly, GLUT1 is the predominantly expressed glucose transporter on tumor cells and its overexpression has been associated with poor prognosis^{12,13}. GLUT1 facilitated uptake of glucose to tumor cells enables their infinite growth, invasion and metastasis making them susceptible to GLUT1 inhibition¹⁴. Inhibition of GLUT1 expression or glucose deprivation of cancer cells has been shown to suppress cell growth in vitro and tumor growth in vivo validating targeting of this transporter in cancer therapy¹⁵⁻¹⁷.

GLUT1 high expression also plays a role in other diseases. Infectious agents, such as malaria parasite Plasmodia, rely on glucose consumption for growth and expansion¹⁸. GLUT1 inhibitors disclosed herein will also be useful for inhibiting infectious diseases. In diabetes mellitus, hypoglycemia is a serious side effect of insulin therapy which could be mitigated by GLUT1 inhibiting antibody and insulin fusion constructs¹⁹. In diabetic hyperglycemia on the other hand, dangerously high uptake of glucose can be blocked with GLUT1 inhibitors to reduce the occurrence of damages to tissues such as diabetic retinopathy²⁰.

### SUMMARY OF THE INVENTION

The invention provides multitude of antibodies binding to SLC2A1 and blocking glucose uptake. Blocking antibodies can be used therapeutically as single agents to inhibit glucose uptake. In cancer therapy, anti-SLC2A1 inhibiting antibodies can be used in combination with other drugs to reduce cancer cell fitness and disease burden even more efficiently.

Accordingly, it is an object of the invention to provide SLC2A1 antibodies or antigen binding fragments thereof that block glucose uptake.

It is a further object of the invention to provide methods of inhibiting glucose uptake in a patient comprising administering one or more anti-SLC2A1 antibodies or antigen binding fragments to the patient, wherein in a subset of the tumor cells glucose uptake is inhibited.

It is an additional object of the invention to provide methods of reprogramming tumor cell metabolism by blocking glucose uptake and shifting metabolism towards e.g. oxidative phosphorylation by providing one or more anti-SLC2A1 antibodies or binding fragments thereof in vitro to the tumor cell culture.

It is a further object of the invention to provide methods of reprogramming tumor cell metabolism in a patient by administering one or more anti-SLC2A1 antibodies or binding fragments thereof to the patient, wherein in a subset of the tumor cells' glucose uptake inhibition shifts metabolism towards e.g. oxidative phosphorylation.

It is an additional object of the invention to provide methods of inhibiting cancer cell metabolism in vitro or in vivo by blocking glucose uptake with one or more anti-SLC2A1 antibodies or binding fragments thereof, and inhibiting oxidative phosphorylation with metabolic regulators, such as complex I inhibitors metformin, phenform and IACS-010579.

It is a further object of the invention to provide methods of inhibiting cancer cell metabolism in a patient comprising administering one or more anti-SLC2A1 antibodies or binding fragments thereof simultaneously or sequentially with metabolic regulators, such as complex I inhibitors metformin, phenform and IACS-010579 to the patient, wherein in a subset of the tumor cells glucose uptake inhibition with concurrent metabolic pathway inhibition leads to reduced fitness of the cancer cells.

It is an additional object of the invention to provide methods of treating cancer in a patient, comprising administering one or more anti-SLC2A1 antibodies or binding fragments thereof to the patient, wherein said cancer is treated.

It is a further object of the invention to provide methods of treating infectious agents, such as malaria, in a patient, comprising administering one or more anti-SLC2A1 antibodies or binding fragments thereof to the patient, wherein said infectious agent is treated.

It is an additional object of the invention to provide methods of treating diabetes side effects in a patient, comprising administering one or more anti-SLC2A1 antibodies or binding fragments thereof to the patient, wherein said diabetes side effects are treated.

It is an additional object of the invention to provide methods as outlined above wherein the anti-SLC2A1 antibodies or binding fragments thereof comprising amino acids of one or more complementary determining regions (CDRs) selected from the group consisting of:
a heavy chain CDR1 comprising amino acids selected from the group consisting of SEQ ID 41-80 and SEQ ID NO:337, a heavy chain CDR2 comprising amino acids selected from the group consisting of SEQ ID 81-120 and SEQ ID NO:339, a heavy chain CDR3 comprising amino acids selected from the group consisting of SEQ ID 121-160 and SEQ ID NO:340; and
   a light chain CDR1 comprising amino acids selected from the group consisting of a SEQ ID NO: 201-240 and SEQ ID NO: 342; a light chain CDR2 comprising amino acids selected from the group consisting of SEQ ID NO 241-280, and SEQ ID NO:343; and a light chain CDR3 comprising amino acids selected from the group consisting according of SEQ ID NO: 281-320 and SEQ ID NO:344.

It is a further object of the invention to provide methods as outlined above wherein the anti-SLC2A1 antibody comprises VL amino acid sequence selected from the group consisting of SEQ ID NO:1-40 and SEQ ID NO:341, and VH amino acid sequence selected from the group consisting of SEQ ID NO: 161-180 and SEQ ID NO:337.

It is a further object of the invention to provide methods of diagnosing cancer comprising a) contacting a tissue from a patient suspected to have a cancer with at least one anti-SLC2A1 antibody or binding fragment thereof; and b) determining the presence of over-expression of SLC2A1 in the tissue as an indication of the presence of cancer. The anti-SLC2A1 antibody can be as described herein and as outlined above.

### BRIEF DESCRIPTION OF THE DRAWINGS

FIG. 1. ELISA-based SLC2A1 VLP binding assay and EC50 values of anti-SLC2A1 antibodies. Shown are results from n=3 independent experiments±SEM. ELISA-based binding assays to estimate the binding affinities of anti-SLC2A1 antibodies to SLC2A1 pseudotyped VLPs. The figure shows binding of six different antibodies and their respective effective concentration 50 (EC50) values. All antibodies bind to SLC2A1 on VLPs with low nanomolar EC50.

FIG. 2.A, B. Flow cytometry-based HepG2 (A) and HEPG (B) wild type (wt) and SLC2A1 knock-out (KO) cell binding assay. MFI ratio-mean fluorescent intensity ratio. Shown are results from n=3 independent experiments±SEM. Anti-SLC2A1 antibody binding to HepG2 wild type (wt) and SLC2A1 knock-out (KO) cell lines was evaluated by flow cytometry. Anti-SLC2A1 antibodies bind specifically to wt and not to KO HepG2 cells.

FIG. 3.A,B,C. ELISA-based evaluation of anti-SLC2A1 antibody binding to VLPs carrying SLC2A2, SLC2A3, SLC2A4. (A) 2E5 #22, (B) 6F12 #33 and (C) R14 #54 antibody ELISA-based glucose transporter binding comparison. Shown are results from one to two representative experiments with n=2 technical repetitions±SEM.

FIG. 4. A, B. Deoxyglucose uptake in the presence of anti-SLC2A1 antibodies or BAY-876 glucose uptake inhibitor. Shown are results from one to three representative experiments with n=2 technical repetitions±SEM. (A) Concentration dependent decrease in glucose uptake is observed after 2-hour incubation with anti-SLC2A1 antibodies or BAY-876.

FIG. 5. Anti-SLC2A1 antibody single agent effect on MCF7 cell confluence. Shown are results from n=3 independent experiments±SEM. Inhibition of MCF7 breast cancer cell proliferation in the presence of anti-SLC2A1 antibodies. Decrease in cell confluence is detected after 24 hours of incubation.

FIGS. 6-8 show inhibition of MCF7 breast cancer cell proliferation in the presence of anti-SLC2A1 antibodies and complex I inhibitors metformin (FIG. 6), phenformin (FIG. 7) and IACS-010759 (FIG. 8). A complete and synergistic MCF7 cell growth inhibition is observed when cells are co-treated with anti-SLC2A1 antibodies and complex I inhibitors:

FIG. 6. Anti-SLC2A1 antibody effect in combination with metformin on MCF7 proliferation. Shown are results from one representative experiment with n=4 technical repetitions±SEM.

FIG. 7. Anti-SLC2A1 antibody effect in combination with phenformin on MCF7 proliferation. Shown are results from n=3 independent experiments±SEM.

FIG. 8. Anti-SLC2A1 antibody effect in combination with IACS-010759 on MCF7 proliferation. Shown are results from n=4 independent experiments±SEM.

FIG. 9. Comparison of different anti-SLC2A1 antibody clones in their effect on MCF7 proliferation in combination with phenformin. Dashed line indicates the average MCF7 normalized confluence for untreated samples (UT). Shown are results from one representative experiment with n=3 technical repetitions±SEM. Selection of anti-SLC2A1 antibodies with potent synergistic effect with phenformin co-treatment. Antibodies 2E5 #22, 5G5 #30, 6F12 #33 and 7C3 #39 completely inhibit MCF7 cell growth concentration dependently when combined with phenformin.

FIG. 10. IC50 for anti-SLC2A1 antibody and 130 μM phenformin co-treatment on MCF7 proliferation. Shown are results from two individual experiments with n=2 technical repetitions±SEM. Anti-SLC2A1 inhibit MCF7 cell line growth concentration dependently in the presence of 130 μM phenformin.

FIG. 11 A, B, C. Anti-SLC2A1 antibody effect in combination with IACS-010759 on prostate cancer (A) and pancreatic cancer (B, C) cell line proliferation. Shown are results from one representative experiment with n=2 technical repetitions. Validation of anti-SLC2A1 and complex I inhibitor synergistic growth inhibition. Anti-SLC2A1 antibodies drastically inhibit PC3 prostate cancer and pancreatic cancer cell line PANC1 and MIAPACA2 growth when combined with IACS-010759.

FIG. 12A, B, C. Antitumor efficacy against established MiaPaca2 xenograft tumors. (A) measurements of the tumor volume (A), of the body weight (B), as well as antibody serum concentration (C) over time. Shown are results from one experiment with n=10 animals in each group.

### DETAILED DESCRIPTION OF THE INVENTION

### Definitions

As used here, “antibody” is a polypeptide that specifically binds and recognizes an antigen or an antigenic fragment thereof.

A “monoclonal antibody” is an antibody obtained from a population of substantially homogeneous antibodies, i.e., the individual antibodies of the population are identical except for possible naturally occurring mutations that may be present in minor amounts. Monoclonal antibodies are highly specific, being directed against a single antigenic epitope.

The term “disease” refers to any alternation in state of the body or of some of the organs, interrupting or disturbing the performance of the functions and/or causing symptoms such as discomfort, dysfunction, distress, or even death to the person afflicted or those in contact with a person. Cancer or tumor is within the definition of disease.

As used herein, the terms “treat,” “treatment,” “treating,” refer to therapeutic treatments, wherein the object is to reverse, alleviate, ameliorate, inhibit, slow down or stop the progression or severity of a disease, including cancer.

A treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptoms, diminishment of extent of disease, stabilized (i.e., not worsening) state of disease, delay or slowing of disease progression, amelioration or palliation of the disease state, remission (whether partial or total), and/or decreased mortality.

As used herein, the term “prevent” or “prevention” refers to stopping, hindering, and/or slowing down a disease. In one embodiment, “prevent” is synonymous with “inhibit”.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

As used herein, a “subject” means a mammal, preferably a human. The terms, “individual,” “patient” and “subject” are used interchangeably herein.

The terms “increased”, “increase”, or “enhance” are all used herein to generally mean an increase by a statically significant amount; the terms “increased”, “increase”, or “enhance”, mean an increase of at least 10% as compared to a 20 reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about 25 a 4-fold, or at least about a 5-fold or at least about a 10-fold increase, or any increase between 2-fold and 10-fold or greater as compared to a reference level.

The terms “decrease”, “reduce”, “reduction”, or “inhibit” are all used herein generally to mean a decrease by a statistically significant amount. For example, “decrease”, 30 “reduce”, “reduction”, or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level or non-detectable level as compared to a reference level), or any decrease between 10-100% as compared to a reference level. In the context of a marker or symptom is meant a statistically significant decrease in such level. The decrease can be, for example, at least 10%, at least 20%, at least 30%, at least 40% or more, and is preferably down to a level accepted as within the range of normal for an 5 individual without a given disease.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “in combination” refers to the use of more than one prophylactic and/or therapeutic agent simultaneously or sequentially and in a manner that their respective effects are additive or synergistic.

“Composition” as used herein may be a single or a combination of antibodies or antigen binding fragments thereof disclosed herein, which can be the same or different, in order to prophylactic or therapeutic treatment. Such combinations can be selected according to the desired immunity or effect. The antibody-based pharmaceutical composition of the present invention may be formulated by any number of strategies known in the art (e.g., see McGoff and Scher, 2000, Solution Formulation of Proteins/Peptides: In McNally, E. J., ed. Protein Formulation and Delivery. New York, N.Y.: Marcel Dekker; pp. 139-158; Akers and Defilippis, 2000, Peptides and Proteins as Parenteral Solutions. In: Pharmaceutical Formulation Development of Peptides and Proteins. Philadelphia, Pa.: Talyor and Francis; pp. 145-177; Akers, et al., 2002, Pharm. Biotechnol. 14:47-127).

A growing body of evidence supports the notion that cancer cell altered metabolism could be exploited as therapeutic target. The aberrant glucose metabolism of human tumors is known as the Warburg effect which is characterized by a significantly elevated rate of glucose consumption and lactate excretion even in the presence of oxygen²¹. The elevated levels of glucose consumption sustains high cell proliferation as glycolytic intermediates are used in various pathways to facilitate the biosynthesis of new macromolecules and organelles²². To enable the uptake of copious amounts of glucose, the expression of glucose transporters is often upregulated on tumor cells^{13,23,24}. The increased uptake and utilization of glucose has also been observed in clinical setting where glucose uptake is visualized by using labeled glucose analogs GLUT1 inhibition can result in blocking the metabolic pathways downstream from glucose and restrict the proliferation of glycolysis dependent cancer cells. Small molecule inhibitors of GLUT1 have been developed but they lack specificity for GLUT1²⁶⁻²⁸. Targeting other group I glucose transporters is not preferred as they carry important functions in central processes in the body. Inhibiting GLUT1 function with monoclonal antibodies could enable much higher specificity than small molecule drugs.

Cancers that are highly reliant to glycolysis would be especially sensitive to GLUT1 inhibition. For example, high expression of SLC7A11 sensitizes cancer cells to acute death during glucose limitation^{29,30} and inactivating mutations in the TCA cycle proteins or OXPHOS can also lead to greater glucose dependency³¹⁻³⁴. However, some tumors have inherent metabolic plasticity and can use other energy-generating pathways than glycolysis to sustain their energetic needs.

It has been shown that inhibiting glycolysis alone induces a metabolic shift toward mitochondrial-dependent oxidative phosphorylation (OXPHOS)³⁵. Co-targeting glycolysis with anti-GLU1 therapeutic monoclonal antibodies and OXPHOS may hold greater promise to specifically restrict the growth of such tumor cells.

### Combination Therapies

The compounds of this invention can be administered as the sole pharmaceutical agent or in combination with one or more other pharmaceutical agents where the combination causes no unacceptable adverse effects. The present invention relates also to such combinations. Possible combinatorial approaches of the compounds include standard chemotherapy options, but also existing and novel targeted therapy and immunotherapy combinations (for example angiogenesis inhibitors, receptor tyrosine kinase inhibitors, CTLA4 and PD-1/PD-L1 inhibitors). Additionally, compounds affecting metabolic pathways can be added to the composition, including but not limited to complex I inhibitors such as metformin, phenformin and IACS-010759.

Generally, the use of systemic oncologic agents in combination with a compound or composition of the present invention will serve to:
(1) yield better efficacy in reducing the growth of a tumor or even eliminate the tumor as compared to administration of either agent alone,
(2) provide for the administration of lesser amounts of the administered chemotherapeutic/targeted/immunogenic agents,
(3) provide for a treatment that is well tolerated in the patient with fewer deleterious pharmacological complications than observed with single agent therapies and certain other combined therapies,
(4) provide for treating a broader spectrum of different cancer types in mammals, especially humans,
(5) provide for a higher response rate among treated patients,
(6) provide for a longer survival time among treated patients compared to standard treatments, (7) provide a longer time for tumor progression, and/or
(8) yield efficacy and tolerability results at least as good as those of the agents used alone, compared to known instances where other cancer agent combinations produce antagonistic effects.

The compounds and compositions of this disclosure are expected to be effective as therapeutic agents in treating various tumor types. In vitro and in vivo experiments will be conducted with breast cancer, colorectal cancer, and pancreatic cancer all of which are expected to have response to the compounds and compositions. Further the compositions and compounds may be used in treating a number of other cancer types, such as, but not limited to lung cancer, hepatocarcinoma, biliary cancer, cervical cancer.

Furthermore, the compounds and compositions of this disclosure are expected to be effective in treating infectious diseases, such as malaria, as well as treating diabetes side effects in a patient.

Even further uses of the compounds and compositions of this disclosure are methods of diagnosing cancer in a patient by contacting a tissue from a patient with an anti-SLC2A1 antibody; and determining the presence of over-expression of SLC2A1 in the tissue as an indication of the presence of cancer.

### DETAILED DESCRIPTION OF THE INVENTION

### Immunization of Chickens, Discovery and Isolation of SLC2A1 Binding Monoclonal Antibody Using Hybrifree Technology

Immunization, discovery and isolation of monoclonal antibody recognizing SLC2A1 protein was conducted using Hybrifree technology (Kivi G, et al., BMC 15 Biotechnol. 2016; 16:2; incorporated herein by reference). Approximately 5-8-month-old Brown Nick chickens were immunized 5 times with 0.6 mg of SLC2A1 DNA expression vector per injection in combination with electroporation (EP). This was followed by one final boost with SLC2A1 pseudotyped VLPs. The chickens were immunized after every 2-2.5 weeks, intramuscularly and intradermally. 2.5 weeks after 5^{th} immunization chickens were boosted with SLC2A1 pseudotyped VLPs intramuscularly, intravenously or intradermally. After confirmation of antigen-specific antibody response in serum, spleens were collected 2-4 days post final immunization. The animals were anesthetized, spleen was removed and stored on ice until treatment (within one hour). For the preparation of cell homogenate, the spleen was homogenized in ice-cold PBS using a 40 m cell dissociation sieve. Cells were precipitated and frozen in 1 ml cryovials using ice-cold freezing medium [fetal bovine serum, (FBS)+10% DMSO]. For antibody isolation, frozen spleen cell suspension was thawed, washed and collected into 10 ml of RPMI1640 supplemented with penicillin/streptomycin and 10% of FBS. Then the cells were seeded into a 100 mm cell culture dish and incubated ˜1 h at 37° C. in an 8% CO2 atmosphere. Then, free-floating cells (the fraction enriched for B-cells and separated from plastic-adherent cells, e.g. macrophages) were collected, viable cell count was determined, and the cells were transferred into the capture medium (RPMI1640 supplemented with 0.5% BSA and 0.1% NaN3). For capture or panning of the B-cells that express SLC2A1 protein specific antibodies on their surface, MaxiSorp™ surface 96-wells (Thermo Fisher Scientific, US) were coated with SLC2A1 pseudotyped VLPs and blocked for 1 h with 2% BSA in PBS. One hundred microliters of cell suspension containing 4×10⁴ live cells in capture medium were loaded into a single well. The plate was centrifuged (200×g, 5 min) and incubated for 45-60 min. The medium was discarded, and loosely attached cells were removed by washing 4-5 times with PBS. Finally, the plastic-bound cells were lysed and subjected to total RNA isolation and cDNA synthesis using oligo-T primer. The cDNA synthesis was conducted using SuperScript™ IV First-Strand Synthesis System (Thermo Fischer Scientific Catalog number: 18091050) and oligo d(T)20 primer supplied with the kit. The cDNAs of antibody VH and VL regions were amplified using forward primers that bind to FR1 regions of VH or VL_lambda light chain coding sequences and reverse primer that binds to the junction area between variable and constant region encoding sequence of the chicken IgY heavy chain and lambda light chain, respectively.

Chicken HC forward primer was according to SEQ ID NO:321

(GTGACCACAGGCGTCCACAGCGCCGTGACGTTGGACGAGTCCG)

Chicken HC reverse primer was according to SEQ ID NO:322

(CACGCTAGGTCCCTTGGTCGAAGCGGAGGAGACGATGACTTCGGTC)

Chicken LC-lambda forward primer was according to SEQ ID NO: 323

(GGCTGACAGACGCCAGGTGCGCGCTGACTCAGCCGTCCTCG)

Chicken LC reverse primer was according to SEQ ID NO: 324

(GGAGCGGCCTTAGGCTGGCCTAGGACGGTCAGGGTTGTCCC)

Using the ligase independent cloning strategy, VH and VL PCR products retrieved from the same capture reaction were both exactly joined with the promoter as well as secretion leader peptide cDNA at the 5′ end and with constant domain cDNA at the 3′ end. The reaction creates just natural joining between variable and constant domain in the human IgG1 heavy lambda light chain, respectively. The final product resulting from the cloning reaction is the pQMCF IgG shuttle expression vector containing ampicillin resistance gene for selective growth of transformed E. coli and separate mammalian expression cassettes for the IgG1 heavy and light chain, respectively. Transformation of the cloning reactions to competent E. coli cells and the bacterial culture was propagated in liquid media. Then purification of the plasmid DNA products from the propagated bacteria resulted in library pools of the antibody expressing vectors. In principle, such library pools are VH/VL combinatory libraries of limited numbers of VH and VL sequences retrieved from the same capture reaction and linked pairwise in the single expression vector molecule. The efficiency of the antigen-specific IgG reconstruction from VH and VL combinations was initially analyzed via the transfection of library pools. The DNA was transfected into CHO cells, and 48-72 h later the culture supernatants were assessed by ELISA for the secretion of IgG molecules that specifically recognizing SLC2A1 protein (VLPs), thus indicating the presence of the desired VH/VL combinations in the library. Next, the library pools that showed a clearly positive signal were split to individual clones by back-transformation into competent E. coli cells and picking individual bacterial colonies—each containing single type of the plasmid with unique VH and VL combination. Then, specific SLC2A1-binding determination by ELISA was repeated using the supernatants of CHO cells transfected with plasmid DNA preparations derived from single clones instead of library pools. Finally, the VH and VL sequences were identified by sequencing the VH and VL insertions of the positive plasmid clones.

### Affinity Maturation Library and Oligonucleotide Design

Affinity maturation library was designed based on structural analysis and rational approach. Four oligonucleotides annealing to H1, H2, H3 and L3 CDR regions containing different number of randomized codons were synthesised. Oligonucleotide randomization was performed using non-equimolar degenerate oligonucleotides that combine mixture of nucleotides at specific positions during the DNA synthesis (Integrated DNA Technologies) and mutagenic oligonucleotides were generated.

The mutated oligonucleotides were used to generate affinity maturation library with randomized CDR regions H1, H2, H3, and L3 using Kunkel mutagenesis protocol (Kunkel, 1985). To force mutations and avoid obtaining the wt #33 Fab displayed on phage, two STOP codons (TAA and TGA) in tandem were introduced in the template phagemid in CDR H2 by Kunkel mutagenesis. These STOP codons substituted two amino acids in CDR H2 to generate a stop template, resulting in non-functional Fabs. STOP template was generated with AM_H2_Stop_Short oligonucleotide according to SEQ ID NO: 332.

CTGGAATTCGTCGCAGCTATTTAATGATGGACAGCCTACGCGAC

The ccc-dsDNA with the desired mutations were electroporated into SS320 E. Coli cells, grown overnight and the phage library was extracted by PEG/NaCl precipitation in the following day. Nunc ImmunoSorp 96-well ELISA plates (Thermo Fisher Scientific, US) were coated with SLC2A1 VLP or BSA in PBS and then washed and blocked with 1% BSA, 0.05% Tween 20. The library was incubated on negative BSA selection plate, after which the library was transferred to the SLC2A1 selection plate for positive selection. Unbound, unspecific, and weakly binding phages were removed with washes and target-bound phages were eluted using 0.1 M HCl followed by neutralisation with 1 M Tris pH 11 buffer. The eluted phages were then collected and used for titration or reinfection. For reinfection, The XL1 Blue cells were infected with phage output and helper phage M13KO7 (New England Biolabs, US) to precipitate the phages on the following day. SLC2A1 specific phages were identified by SLC2A1 VLP ELISA and the VH and VL sequences of the positive phage clones were identified by PCR amplification and sequencing the VH and VL insertions.

PCR amplification forward primer was according to SEQ ID NO:333.
TGTAAAACGACGGCCAGTCTATTGCTACAAATGCCTATGCATCC

PCR amplification reverse primer was according to SEQ ID NO:334.
CAGGAAACAGCTATGACCCACCGGTTCGGGGAAGTAG

Sequencing forward primer was according to SEQ ID NO:335.
TGTAAAACGACGGCCAGT

Sequencing reverse primer was according to SEQ ID NO:336
CAGGAAACAGCTATGACC

### ELISA-Based VLP Binding Assay

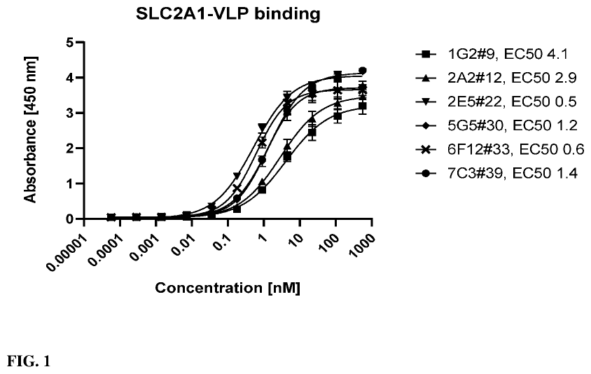

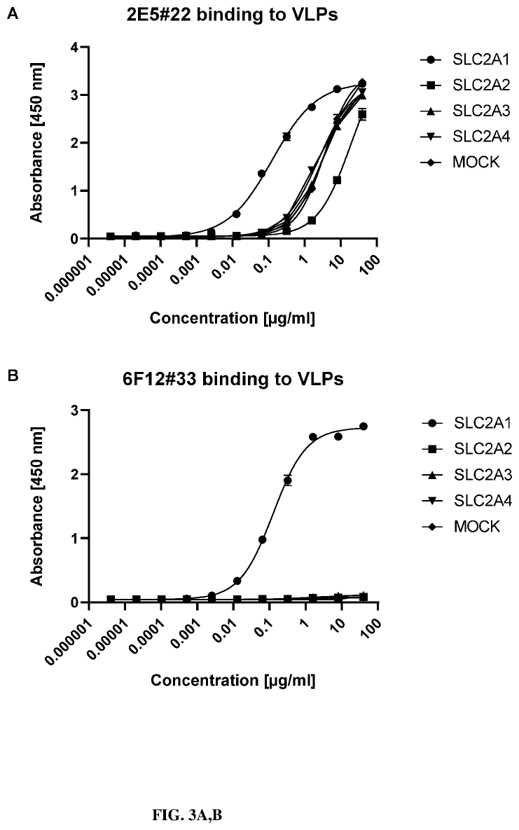

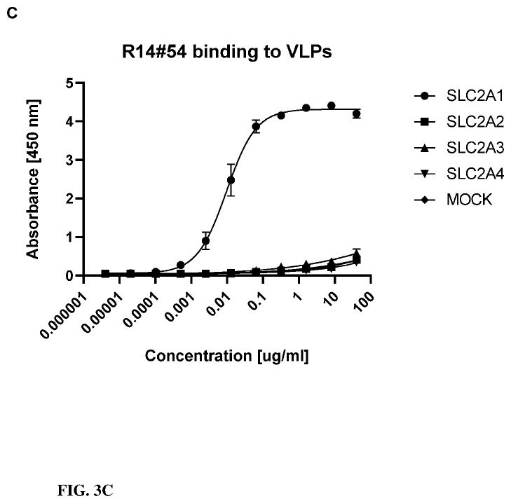

An ELISA-based VLP binding assay was carried out to compare the EC50 values of antibody clones (FIG. 1). Similarly, ELISA based VLP binding assay was performed to compare the binding of the antibodies to different glucose transporters (FIG. 3). MaxiSorp™ high-capacity binding ELISA plate (Thermo Fisher Scientific, US) were coated with 5 μg/mL SLC2A1, SLC2A2, SLC2A3, SLC2A4 pseudotyped VLPs or MOCK VLPs in PBS overnight. The plate was washed and blocked for 1 h with 0.05% Tween 20, 2% BSA in PBS. Serial dilutions of antibodies were incubated on a washed plate for 1 hour, followed by a 1-hour incubation with goat anti-human IgG horse radish peroxidase (HRP) conjugated (Invitrogen) antibody. TMB (3,3′,5,5′-Tetramethylbenzidine) Solution VII (Biopanda Diagnostics) was used as a substrate for HRP and 0.5 M H₂SO₄ was used as stop solution. After every step, the wells were washed with 0.05% Tween 20, 0.1% Proclin 300 PBS solution. Absorption at 450 nm was measured with Thermo Scientific™ Multiskan™ FC Microplate Photometer. Exemplary wise used antibodies 1G2 #9, 2A2 #12, 2E5 #22, 5G5 #30, 6F12 #33, 7C3 #39 all bound SLC2A1 VLPs with low nanomolar EC50 values (FIG. 1). Exemplary wise used antibodies 2E5 #22 and 6F12 #33 both did not bind to SLC2A2, SLC2A3 and SLC2A4 VLPs more than to MOCK VLPs (FIG. 3).

### Flow Cytometry-Based Cell Binding Assay

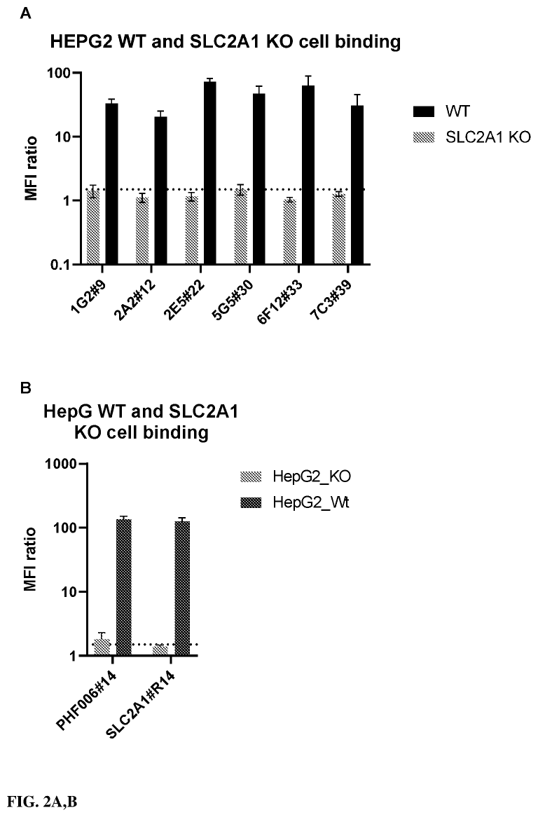

Flow cytometry-based HepG2 wild type (wt, Abcam, ab275467) and SLC2A1 knock-out (KO) cell (Abeam, ab280797) binding assay was used to demonstrate the specific binding of the anti-SLC2A1 antibodies to SLC2A1 membrane protein (FIG. 2). For this, 0.1×10⁶ cells were incubated in the presence of 100 nM anti-SLC2A1 antibody solutions in flow cytometry buffer (1% FBS, 1 mM EDTA in PBS) on ice. This was followed by an incubation with a secondary goat anti-human IgG AF488 antibody (Jackson Immuno Research). The mean fluorescent intensity (MFI) was measured in FITC channel with Becton, Dickinson and Company Accuri C6 Plus flow cytometer and MFI ratio was calculated by dividing the MFI of each antibody to anti-HEL isotype (Icosagen Cell Factory) control staining MFI. MFI ratio above 1.5 is considered as binding. All evaluated antibodies did not bind to HepG2 SLC2A1 KO as the MFI ratios were below 1.5. However, all antibodies bound to HepG2 wt cells with MFI ratios well above 1.5. The results indicate that a specific binding to SLC2A1 membrane protein is observed.

### 2-Deoxyglucose Uptake Assay

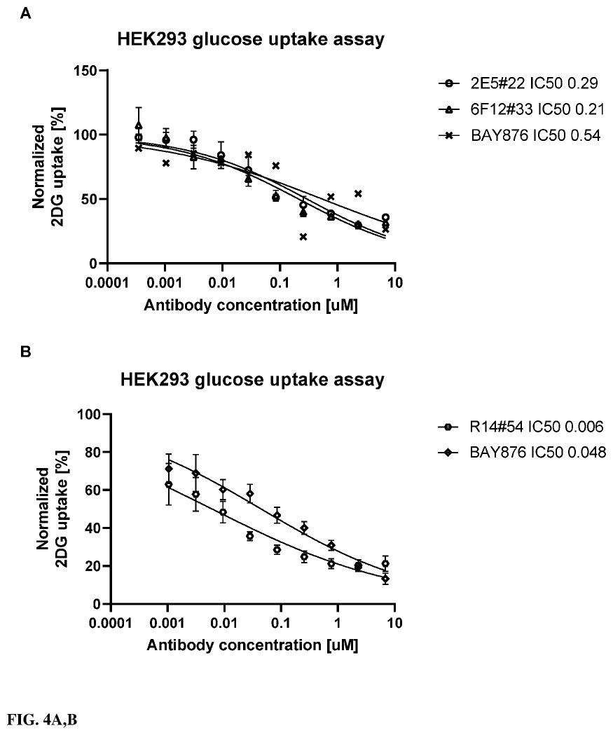

To evaluate if the described anti-SLC2A1 antibodies inhibit glucose uptake, a commercial Glucose Uptake-Glo Assay kit (Promega) was utilized according to manufacturer's instructions. In short, HEK-293-ALL cells were transfected with human SLC2A1 expression plasmid and were incubated at 37° C. overnight in the presence of 5% CO2. Cells were washed with 0.5% BSA PBS twice and 7000 cells were plated per well into white clear bottom 96-well plates in 0.5% BSA PBS in the presence of 0.35 nM to 6.89 μM anti-SLC2A1 antibodies or glucose uptake inhibitor BAY-876 (CaymanChem). Cells were incubated for 2 hours at room temperature. Glucose uptake was initiated by the addition of 1 mM 2-deoxyglucose (2DG) and was allowed to proceed for 20 min at 37° C. in the presence of 5% CO₂. After adding STOP and neutralization reagents included in the kit, luminescence was measured using GloMax Explorer microplate reader (Promega). Nonspecific luminescence was measured in lysates from cells without (2DG) being added. Data was normalized to nonspecific luminescence and to the average value of luminescence in the presence of no inhibitors. A strong concentration dependent decrease in glucose uptake was observed after adding anti-SLC2A1 antibodies; exemplary wise antibodies 2E5 #22 and 6F12 #33 or BAY-876 were used to show results in FIG. 4.

### Cell Proliferation Inhibition Assays

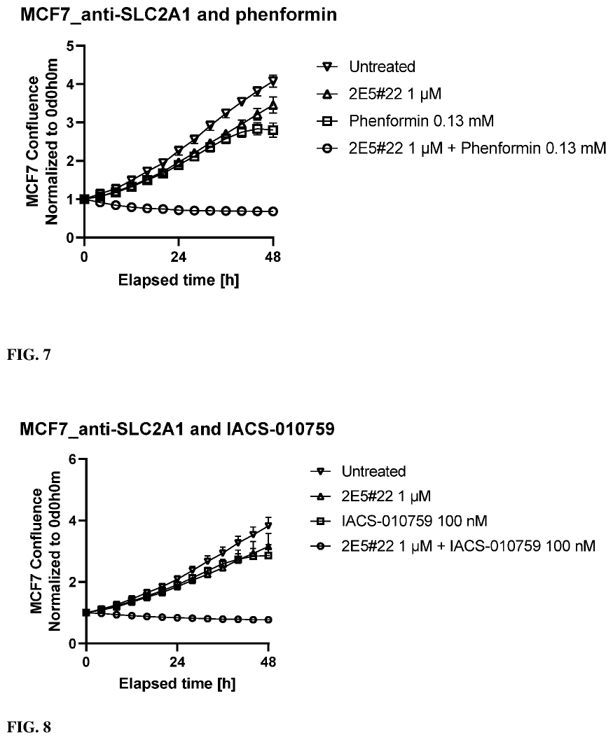

Incucyte S3 Live cell analysis instrument and Cell-by-Cell analysis software module (Sartorius) were used to evaluate the effect of anti-SLC2A1 antibodies on cancer cell proliferation label-free (FIGS. 5-11). MCF7, PC3 or PANC1 or MIAPACA2 cells were plated on flat bottom tissue culture 96-well plate in Plasmax medium (Ximbio) supplemented with 2.5% FBS. Cells were incubated overnight and 0.02 nM to 13 μM anti-SLC2A1 antibodies, anti-HEL isotype control, PBS or BAY-876 were added to the cells alone or in combination with metformin (CaymanChem), phenformin (Sigma Aldrich) or IACS-010759 (CaymanChem). Cell confluence or object count was measured for 24 to 96 hours with Incucyte S3 and normalized to timepoint 0 h or 4h. anti-SLC2A1 antibodies inhibit cancer cell proliferation alone and especially in combination with complex-I inhibitors metformin, phenformin and IACS-010759. Exemplary antibodies 1G2 #9, 2A2 #12, 2E5 #22, 5G5 #30, 6F12 #33, 7C3 #39 and R14 #54 were used in proliferation assays.

### Antitumor Efficacy Against MiaPaca2 Xenograph Tumors

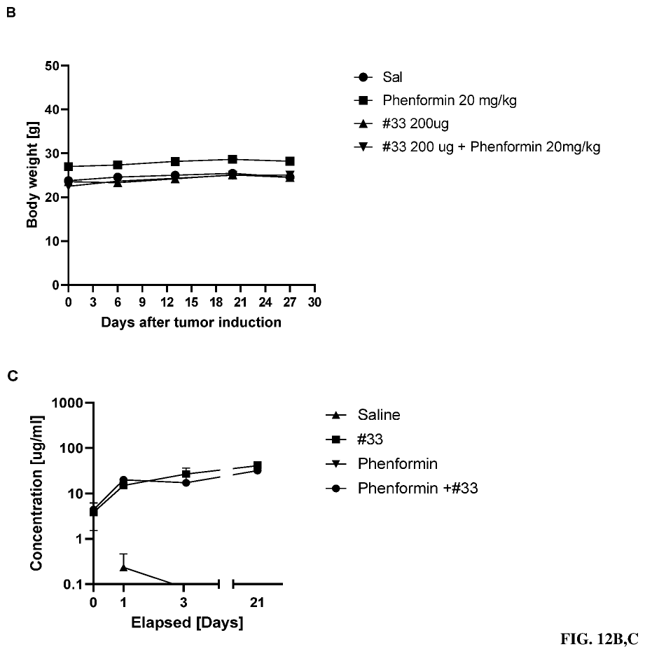

Hsd:Athymic Nude-Foxn1^{nu} mice (10 per group) were inoculated with 5e6 MiaPaca2 cells/mouse subcutaneously. Tumors were allowed to form to a size of 100 mm3 and then, IP treatments of 6F12 #33 200 ug per mouse were started and dosed after every 72 h with or without daily doses of Phenformin 20 mg/kg/IP. FIG. 12A to C shows the results of measurements of the tumor volume over time, measurement of the body weight over time, as well as serum concentration over time. A statistically significant tumor growth inhibition was observed for 6F12 #33 and phenformin cotreatment in comparison to phenformin alone using a mixed-effects model followed by Tukey's post-hoc test. Body weight remained unchanged. Antibody levels in serum monitored by VLP ELISA stayed stable during the experiment.

### Characterization of Antibody Clones

The antibody clones obtained are described in the below table in terms of VH and VL sequences, as well as heavy chain and light chain CDRs.

TABLE 1
Characterization of antibody clones

| ANTIBODY | | | | | | | | |
|---|---|---|---|---|---|---|---|---|
| NAME | VH SEQUENCE | | H-CDR1 | | H-CDR2 | | H-CDR3 | |
| 1G2_9 | AVTLDESGGGLQTPGGALSLVCKAS | SEQ ID NO: 1 | GFTFSTKG | SEQ ID NO: 41 | ISSMRRYT | SEQ ID NO: 81 | ARDATYCSWTTSTCGWS | SEQ ID NO: 121 |
| | GFTFSTKGMGWVRQTPGKGLEWV | | | | | | GGTIDA | |
| | AGISSMRRYTYYAPAVKGRATISRDN | | | | | | | |
| | GQSTVRLQLSSLRAEDSGTYYCARDA | | | | | | | |
| | TYCSWTTSTCGWSGGTIDAWGHGT | | | | | | | |
| | EVIVSS | | | | | | | |
| 2A2_12 | AVTLDESGGGLQTPGGALSLVCKGS | SEQ ID NO: 2 | GFTFSTKG | SEQ ID NO: 42 | ISSMSRYT | SEQ ID NO: 82 | ARDATYCSWTASTCGWS | SEQ ID NO: 122 |
| | GFTFSTKGMMWVRQAPGKGLEWV | | | | | | GGTIDA | |
| | AGISSMSRYTYYAPAVKGRATISRDN | | | | | | | |
| | GQSAVRLQLSSLRAEDTGTYYCARD | | | | | | | |
| | ATYCSWTASTCGWSGGTIDAWGH | | | | | | | |
| | GTEVIVSS | | | | | | | |
| 2E5_22 | AVTLDESGGGLQTPGGALSLVCKAS | SEQ ID NO: 3 | GFTFSTKG | SEQ ID NO: 43 | ISSMRRYT | SEQ ID NO: 83 | ARDATYCSWTTSTCGWS | SEQ ID NO: 123 |
| | GFTFSTKGMGWVRQTPGKGLEWV | | | | | | GGTIDA | |
| | AGISSMRRYTYYAPAVKGRATISRDN | | | | | | | |
| | GQSTVRLQLSSLRAEDSGTYYCARDA | | | | | | | |
| | TYCSWTTSTCGWSGGTIDAWGHGT | | | | | | | |
| | EVIVSS | | | | | | | |
| 5G5_30 | AVTLDESGGGLQTPGGALSLVCKAS | SEQ ID NO: 4 | GFTFSSHA | SEQ ID NO: 44 | ISSSSGSWT | SEQ ID NO: 84 | AKAAGSACCYAGSIDA | SEQID NO: 124 |
| | GFTFSSHAIFWVRQAPDKGLEFVAAI | | | | | | | |
| | SSSSGSWTAYATVVKGRATISRDNG | | | | | | | |
| | QSTVRLOLNNLRAEDTGTYYCAKAA | | | | | | | |
| | GSACCYAGSIDAWGHGTEVIVSS | | | | | | | |
| 6F12_33 | AVTLDESGGGLQTPGGALSLVCKAS | SEQ ID NO: 5 | GFTFSDHA | SEQ ID NO: 45 | ISSGTGSWT | SEQ ID NO: 85 | AKGAGSACCYAGSIDA | SEQ ID NO: 125 |
| | GFTFSDHAIFWVRQAPDKGLEFVAA | | | | | | | |
| | ISSGTGSWTAYATVVKGRATISRDN | | | | | | | |
| | GQSTVRLQLNNLRAADTATYYCAKG | | | | | | | |
| | AGSACCYAGSIDAWGHGTEVIVSS | | | | | | | |
| 7C3_39 | AVTLDESGGGLQTPGGALSLVCKAS | SEQ ID NO: 6 | GFTFSTKG | SEQ ID NO: 46 | ISSMRRYT | SEQ ID NO: 86 | ARDATYCSWTTSTCGWS | SEQ ID NO: 126 |
| | GFTFSTKGMGWVRQTPGKGLEWV | | | | | | GGTIDA | |
| | AGISSMRRYTYYAPAVKGRATISRDN | | | | | | | |
| | GQSTVRLQLSSLRAEDSGTYYCARDA | | | | | | | |
| | TYCSWTTSTCGWSGGTIDAWGHGT | | | | | | | |
| | EVIVSS | | | | | | | |
| 51 | AVTLDESGGGLQTPGGALSLVCKAS | SEQ ID NO: 7 | GFSIRNYN | SEQ ID NO: 47 | ISTAGSYT | SEQ ID NO: 87 | AKHSGPGWYFGSGATAG | SEQ ID NO: 127 |
| | GFSIRNYNMAWVRQAPGKGLEWV | | | | | | LIDA | |
| | GAISTAGSYTSYGPAVKGRATISRDN | | | | | | | |
| | GQSTVRLQLNNLRAEDTGTYYCAKH | | | | | | | |
| | SGPGWYFGSGATAGLIDAWGHGTE | | | | | | | |
| | VIVSS | | | | | | | |
| MD1_1A10 | AVTLDESGGGLQTPGGALSLVCKAS | SEQ ID NO: 8 | GFSIRNYN | SEQ ID NO: 48 | ISTAGSYT | SEQ ID NO: 88 | AKHSGPGWYFGRGASAG | SEQ ID NO: 128 |
| | GFSIRNYNMAWVRQAPGKGLEWV | | | | | | LIDA | |
| | GAISTAGSYTAYAPAVKGRATISRDN | | | | | | | |
| | GQSTVRLQLNNLRAEDTGTYYCAKH | | | | | | | |
| | SGPGWYFGRGASAGLIDAWGHGNE | | | | | | | |
| | VIVSS | | | | | | | |
| MD1_1B6 | AVTLDESGGGLQTPGGALSLVCKAS | SEQ ID NO: 9 | GFTFSSYG | SEQ ID NO: 49 | IRKDGSFT | SEQ ID NO: 89 | ARGGGCSSCDNFAGFIDA | SEQ ID NO: 129 |
| | GFTFSSYGMGWVRQAPGKGLEFVG | | | | | | | |
| | GIRKDGSFTYYGAAVKGRATISRDNG | | | | | | | |
| | QSTVRLQLNNLRAEDTGNYFCARGG | | | | | | | |
| | GCSSCDNFAGFIDAWGHGTEVIVSS | | | | | | | |
| MD1_1C2 | AVTLDESGGGLQTPGGALSLVCKAS | SEQ ID NO: 10 | GFTFSTKG | SEQ ID NO: 50 | ISSMRRYT | SEQ ID NO: 90 | ARDATYCSWTTSTCGWS | SEQ ID NO: 130 |
| | GFTFSTKGMGWVRQTPGKGLEWV | | | | | | GGTIDA | |
| | AGISSMRRYTYYAPAVKGRATISRDN | | | | | | | |
| | GQSTVRLQLSSLRAEDSGTYYCARDA | | | | | | | |
| | TYCSWTTSTCGWSGGTIDAWGHGT | | | | | | | |
| | EVIVSS | | | | | | | |
| MD1_1D8 | AVTLDEAGGGLQTPGGALSLVCKAS | SEQ ID NO: 11 | GFTFSDRG | SEQ ID NO: 51 | ISSGSGSST | SEQ ID NO: 91 | VRGACCSSIDA | SEQ ID NO: 131 |
| | GFTFSDRGIHWVRQAPGKGLEYVAG | | | | | | | |
| | ISSGSGSSTGYGAAVKGRATISRDNG | | | | | | | |
| | QSTVRLOLNNLRAEDTGTYFCVRGA | | | | | | | |
| | CCSSIDAWGHGTEVIVSS | | | | | | | |
| MD1_1F8 | AVTLDESGGGLQTPGGALSLVCKAS | SEQ ID NO: 12 | GFSIRNYN | SEQ ID NO: 52 | ISTAGSYT | SEQ ID NO: 92 | AKHSGRGWYFGRGASAG | SEQ ID NO: 132 |
| | GFSIRNYNMAWVRQAPGKGLEWV | | | | | | LIDA | |
| | GAISTAGSYTAYAPAVKGRATISRDN | | | | | | | |
| | GQSTVRLQLNNLRAEDTGTYYCAKH | | | | | | | |
| | SGRGWYFGRGASAGLIDAWGHGN | | | | | | | |
| | EVIVSS | | | | | | | |
| MD1_2A2 | AVTLDESGGGLQTPGGALSLVCKAS | SEQ ID NO: 13 | GFTFSSYG | SEQ ID NO: 53 | IRKDGSYT | SEQ ID NO: 93 | ARGGGCNTCDNIAGFIDA | SEQ ID NO: 133 |
| | GFTFSSYGMGWVRQAPGKGLEFVA | | | | | | | |
| | GIRKDGSYTYYGAAVKGRATISRDNG | | | | | | | |
| | QSTLRLQLNSLRAEDTATYFCARGG | | | | | | | |
| | GCNTCDNIAGFIDAWGHGTEVIVSS | | | | | | | |
| MD1_2D10 | AVTLDESGGGLQTPGGGLSLVCKAS | SEQ ID NO: 14 | GFTFSSYT | SEQ ID NO: 54 | IANSP | SEQ ID NO: 94 | AKSFDTYGCIYGRCAGRID | SEQ ID NO: 134 |
| | GFTFSSYTMQWVRQAPGKGLEWV | | | | | | T | |
| | AGIANSPYYGAAVQGRATISRDNGQ | | | | | | | |
| | STLRLQLNNLRAEDTATYYCAKSFDT | | | | | | | |
| | YGCIYGRCAGRIDTWGHGTEVIVSS | | | | | | | |
| MD1_2F9 | AVTLDESGGGLQTPGGGLSLVCKAS | SEQ ID NO: 15 | GFSFSSHG | SEQ ID NO: 55 | ISGAGSWT | SEQ ID NO: 95 | AKSSFGCSNSCRNYAGTID | SEQ ID NO: 135 |
| | GFSFSSHGMGWVRQAPGKGLEYVA | | | | | | A | |
| | AISGAGSWTGYGSAVKGRATISRDN | | | | | | | |
| | GQSTMRLQLNNLRAEDTGTYYCAKS | | | | | | | |
| | SFGCSNSCRNYAGTIDAWGHGTEVI | | | | | | | |
| | VSS | | | | | | | |
| S2K_C1 | AVTLDESGGGLQTPGGALSLVCKAS | SEQ ID NO: 16 | GFSISNYN | SEQ ID NO: 56 | ISTGGSYT | SEQ ID NO: 96 | AKHSEGGWYFGSGATAGL | SEQ ID NO: 136 |
| | GFSISNYNMAWVRQAPGKGVEWV | | | | | | IDA | |
| | GAISTGGSYTAYGPAVKGRATISRDN | | | | | | | |
| | GQSTMRLHLNNLRAEDTGTYYCAKH | | | | | | | |
| | SEGGWYFGSGATAGLIDAWGHGTE | | | | | | | |
| | VIVSS | | | | | | | |
| S2K_H11 | AVTLDESGGGLQTPGGALSLVCKAS | SEQ ID NO: 17 | GFSISGYN | SEQ ID NO: 57 | ISTAGSYT | SEQ ID NO: 97 | AKHSGPGWYFGRGSTAG | SEQ ID NO: 137 |
| | GFSISGYNMAWVRQAPGKGLEYVA | | | | | | RIDT | |
| | GISTAGSYTGYAPAVKGRATISRDNG | | | | | | | |
| | QSTVRLQLNNLRAEDTATYYCAKHS | | | | | | | |
| | GPGWYFGRGSTAGRIDTWGHGTEV | | | | | | | |
| | IVSS | | | | | | | |
| L12-1H - | AATLDESGGGLQTPGGALSLVCKAS | SEQ ID NO: 18 | GFTFSDHA | SEQ ID NO: 58 | ISSGTGSWT | SEQ ID NO: 98 | AKGAGSACCYAGSIDA | SEQ ID NO: 138 |
| L12-1L | GFTFSDHAIFWVRQAPDKGLEFVAA | | | | | | | |
| | ISSGTGSWTAYATVVKGRATISRDN | | | | | | | |
| | GQSTVRLQLNNLRAADTATYYCAKG | | | | | | | |
| | AGSACCYAGSIDAWGHGTEVIVSS | | | | | | | |
| E01_R2 | AVTLDESGGGLQTPGGALSLVCKAS | SEQ ID NO: 19 | GFTFSSYG | SEQ ID NO: 59 | IRKDGSTT | SEQ ID NO: 99 | ARGGGCKTCDNIAGFIDA | SEQID NO: 139 |
| | GFTFSSYGMGWVRQAPGRGLEFVA | | | | | | | |
| | GIRKDGSTTYYGAAVKGRATISRDNG | | | | | | | |
| | QSTLRLQLNSLRAEDTATYFCARGG | | | | | | | |
| | GCKTCDNIAGFIDAWGHGTEVIVSS | | | | | | | |
| E01_R3 | AVTLDESGGGLQTPGGALSLVCKAS | SEQ ID NO: 20 | GFSIRNYN | SEQ ID NO: 60 | ISTAGSYT | SEQ ID NO: 100 | AKHSGPGWYFGRGASAGL | SEQ ID NO: 140 |
| | GFSIRNYNMAWVRQAPGKGLEWV | | | | | | | |
| | GAISTAGSYTAYAPAVKGRATISRDN | | | | | | | |
| | GQSTVRLQLNNLRAEDTGTYYCAKH | | | | | | | |
| | SGPGWYFGRGASAGLIDAWGHGTE | | | | | | | |
| | VIVSS | | | | | | | |
| E02_R2_2 | AVTLDESGGGLQTPGGALSVVCKAS | SEQ ID NO: 21 | GLSIRNYN | SEQ ID NO: 61 | ISTAGIYT | SEQ ID NO: 101 | AKHCGPGWYFGSGGYVLG | SEQ ID NO: 141 |
| | GLSIRNYNMAWVRQAPGKGLEWV | | | | | | | |
| | GDISTAGIYTAYAPAVKGRATISRDN | | | | | | | |
| | GQSTVRLQLNNLRAEDTGTYYCAKH | | | | | | | |
| | CGPGWYFGSGGYVLGLIDAWGHGT | | | | | | | |
| | EVIVSS | | | | | | | |
| E03_R3 | AVTLDESGGGLQTPGGALSLVCKAS | SEQ ID NO: 22 | GFSIRNYN | SEQ ID NO: 62 | ISTAGSYT | SEQ ID NO: 102 | AKHSGPGWYFGRGASAGL | SEQ ID NO: 142 |
| | GFSIRNYNMAWVRQAPGKGLEWV | | | | | | | |
| | GAISTAGSYTAYAPAVKGRATISRDN | | | | | | | |
| | GQSTVRLQLNNLRAEDTGTYYCAKH | | | | | | | |
| | SGPGWYFGRGASAGLIDAWGHGTE | | | | | | | |
| | VIVSS | | | | | | | |
| E05_R2_2 | AVTLDESGGGLQTPGGALSLVCKAS | SEQ ID NO: 23 | GFTFSSYG | SEQ ID NO: 63 | IRKDGSFT | SEQ ID NO: 103 | ARGGGCSSCDNFAGFIDA | SEQ ID NO: 143 |
| | GFTFSSYGMGWVRQAPGKGLEFVG | | | | | | | |
| | AIRKDGSFTYYGAAVKGRATISRDNG | | | | | | | |
| | QSTVRLQLSNLRAEDTGTYFCARGG | | | | | | | |
| | GCSSCDNFAGFIDAWGHGTEVIVSS | | | | | | | |
| E06_R2 | AVTLDESGGGLQTPGGALSLVCKAS | SEQ ID NO: 24 | GFTFSSYG | SEQ ID NO: 64 | IRNDGSYT | SEQ ID NO: 104 | ARGGGCNTCDNIAGFIDA | SEQ ID NO: 144 |
| | GFTFSSYGMGWVRQAPGKGLEFVA | | | | | | | |
| | GIRNDGSYTYYGAAVKGRATISRDN | | | | | | | |
| | GQSTLRLQLNSLRAEDTATYFCARG | | | | | | | |
| | GGCNTCDNIAGFIDAWGHGTEVIVS | | | | | | | |
| | S | | | | | | | |
| E06_R2_2 | AVTLDESGGGLQTPGGALSLVCKAS | SEQ ID NO: 25 | GFSIRNYN | SEQ ID NO: 65 | ISTAGSYT | SEQ ID NO: 105 | AKHSGPGWYFGRGASAGL | SEQ ID NO: |
| | GFSIRNYNMAWVRQAPGKGLEWV | | | | | | | 145 |
| | GAISTAGSYTAYAPAVKGRATISRDN | | | | | | | |
| | GQSTVRLQLNNLRDEDTGTYYCAKH | | | | | | | |
| | SGPGWYFGRGASAGLIDAWGHGTE | | | | | | | |
| | VIVSS | | | | | | | |
| E07_R2 | AVTLDESGGGLQTPGGALSLVCKGS | SEQ ID NO: 26 | GFTFSSFH | SEQ ID NO: 66 | ISNDGSS | SEQ ID NO: 106 | ARSPGYCSGGRCYTTSIDA | SEQ ID NO: 146 |
| | GFTFSSFHMFWVRQEPGKGLRWVA | | | | | | | |
| | AISNDGSSAYGSAVKGRATISRDNG | | | | | | | |
| | QGTVRLOLNNLRAEDTATYFCARSP | | | | | | | |
| | GYCSGGRCYTTSIDAWGHGTEVIVSS | | | | | | | |
| E08_R2_2 | AVTLDESGGGLQTPGGALSLVCKAS | SEQ ID NO: 27 | GFSIRNYN | SEQ ID NO: 67 | ISTAGSYT | SEQ ID NO: 107 | AKHSGPGWYFGRGASAGL | SEQ ID NO: 147 |
| | GFSIRNYNMAWVRQAPGKGLEWV | | | | | | | |
| | GAISTAGSYTASAPAVKGRATISRDN | | | | | | | |
| | GQSTVRLQLNNLRAEDTGTYYCAKH | | | | | | | |
| | SGPGWYFGRGASAGLIDAWGHGTE | | | | | | | |
| | VIVSS | | | | | | | |
| F02_R2_2 | AVTSDESGGGLQTPGGALSLVCKAS | SEQ ID NO: 28 | GFSIRNYN | SEQ ID NO: 68 | ISTGGSYT | SEQ ID NO: 108 | AKHSGPGWYFGRGASAGL | SEQ ID NO: 148 |
| | GFSIRNYNMAWVRQAPGKGSEWV | | | | | | | |
| | GTISTGGSYTAYAPAVKGRATISRDN | | | | | | | |
| | GQSTVRLQLNNLRAEDTGTY*CAKH | | | | | | | |
| | SGPGWYFGRGASAGLIDAWGHGTE | | | | | | | |
| | VIVSS | | | | | | | |
| F05_R2 | AVTLDESGGGLQTPGGALSLVCKAS | SEQ ID NO: 29 | GFTFSSYG | SEQ ID NO: 69 | IRNDGSFT | SEQ ID NO: 109 | ARGGGCSSCDNFAGFIDA | SEQ ID NO: 149 |
| | GFTFSSYGMGWVRQAPGKGLEFVA | | | | | | | |
| | AIRNDGSFTYYGAAVKGRATISRDNG | | | | | | | |
| | QSTLRLQLNNLRTEDTGTYFCARGG | | | | | | | |
| | GCSSCDNFAGFIDAWGHGTEVIVSS | | | | | | | |
| F06_R2 | AVTLDESGGGLQTPGGALSLVCKAS | SEQ ID NO: 30 | GFTFSSYG | SEQ ID NO: 70 | IRKDGTYT | SEQ ID NO: 110 | ARGGGCNTCDNIAGFIDA | SEQ ID NO: 150 |
| | GFTFSSYGMGWVRQAPGKGLEFVA | | | | | | | |
| | GIRKDGTYTYYGAAVKGRATISRDNG | | | | | | | |
| | QSTLRLQLNNLRAEDTGTYFCARGG | | | | | | | |
| | GCNTCDNIAGFIDAWGHGTEVIVSS | | | | | | | |
| G06_R3 | AVTLDESGGGLQTPGGALSLVCKAS | SEQ ID NO: 31 | GFSIRNYN | SEQ ID NO: 71 | ISTAGSYT | SEQ ID NO: 111 | AKHSGPGWYFGRGASAGL | SEQ ID NO: 151 |
| | GFSIRNYNMAWVRQAPGKGLEWV | | | | | | | |
| | GAISTAGSYTAYAPAVKGRATISRDN | | | | | | | |
| | GQSTVRLQLNNLRAEDTGTYYCAKH | | | | | | | |
| | SGPGWYFGRGASAGLIDAWGHGTE | | | | | | | |
| | VIVSS | | | | | | | |
| G07_R2 | AVTLDESGGGLQTPGGALSLVCKAS | SEQ ID NO: 32 | GFSIRNYN | SEQ ID NO: 72 | ISTAGSYT | SEQ ID NO: 112 | AKHSGPGWYFGRGASAGL | SEQ ID NO: 152 |
| | GFSIRNYNMAWVRQAPGKGLEWV | | | | | | | |
| | GAISTAGSYTAYAPAVKGRATISRDN | | | | | | | |
| | GQSTVRLQLNNLRAEDTGTYYCAKH | | | | | | | |
| | SGPGWYFGRGASAGLIDAWGHGTE | | | | | | | |
| | VIVSS | | | | | | | |
| G09_R2 | AVTLDESGGGLQTPGGALSLVCKAS | SEQ ID NO: 33 | GFTFSSHA | SEQ ID NO: 73 | ISSSSGSWT | SEQ ID NO: 113 | AKAAGSACCYAGSIDA | SEQ ID NO: 153 |
| | GFTFSSHAIFWVRQAPDKGLEFVAAI | | | | | | | |
| | SSSSGSWTAYATVVKGRATISRDNG | | | | | | | |
| | QSTVRLQLNNLRAEDTGTYYCAKAA | | | | | | | |
| | GSACCYAGSIDAWGHGTEVIVSS | | | | | | | |
| G10_R2 | AVTLDESGGGLQTPGGALSLVCKAS | SEQ ID NO: 34 | GFSISGYN | SEQ ID NO: 74 | ISTAGSYT | SEQ ID NO: 114 | AKHSGPGWYFGRGASAGL | SEQ ID NO: 154 |
| | GFSISGYNMAWVRQAPGKGLEWV | | | | | | | |
| | GAISTAGSYTAYAPAVKGRATISRDN | | | | | | | |
| | GQSTVRLQLNNLRAEDTGTYYCAKH | | | | | | | |
| | SGPGWYFGRGASAGLIDAWGHGTE | | | | | | | |
| | VIVSS | | | | | | | |
| H01_R2 | AVTLDESGGGLQTPGGALSLVCKAS | SEQ ID NO: 35 | GFSIRNYN | SEQ ID NO: 75 | ISTAGSYT | SEQ ID NO: 115 | AKHSGPGWYFGRGASAGL | SEQ ID NO: 155 |
| | GFSIRNYNMAWVRQAPGKGLEWV | | | | | | | |
| | GAISTAGSYTAYAPAVKGRATITRDN | | | | | | | |
| | GQSTVRLQLNNLRAEDTGTYYCAKH | | | | | | | |
| | SGPGWYFGRGASAGLIDAWGHGTE | | | | | | | |
| | VIVSS | | | | | | | |
| H02_R2 | AVTLDESGGGLQTPGGALSLVCKAS | SEQ ID NO: 36 | GFSIRNYN | SEQ ID NO: 76 | ISTAGSYT | SEQ ID NO: 116 | AKHSGPGWYFGRGASAGL | SEQ ID NO: 156 |
| | GFSIRNYNMAWVRQAPGKGLEWV | | | | | | | |
| | GAISTAGSYTAYAPAVKGRATISRDN | | | | | | | |
| | GQSTVRLQLNNLRAEDTGTYYCAKH | | | | | | | |
| | SGPGWYFGRGASAGLIDAWGHGTE | | | | | | | |
| | VIVSS | | | | | | | |
| H03_R3 | AVTLDESGGGLQTPGGGLSLVCKAS | SEQ ID NO: 37 | GFSIRNYN | SEQ ID NO: 77 | ISTAGSYT | SEQ ID NO: 117 | AKHSGPGWYFGRGASAGL | SEQ ID NO: 157 |
| | GFSIRNYNMAWVRQAPGKGLEWV | | | | | | | |
| | GAISTAGSYTAYAPAVKGRATISRDN | | | | | | | |
| | GQSTVRLQLNNLRAEDTGTYYCAKH | | | | | | | |
| | SGPGWYFGRGASAGLIDAWGHGTE | | | | | | | |
| | VIVSS | | | | | | | |
| H05_R2_2 | AVTLDESGGGLQTAGGALSLVCKAS | SEQ ID NO: 38 | GFTFSDHA | SEQ ID NO: 78 | ISSGTGSWT | SEQ ID NO: 118 | AKGAGSACCYAGSIDA | SEQ ID NO: 158 |
| | GFTFSDHAIFWVRQAPDKGLEFVAA | | | | | | | |
| | ISSGTGSWTAYATVVKGRATISRDN | | | | | | | |
| | GQSTVRLQVNNLRAADTATYYCAKG | | | | | | | |
| | AGSACCYAGSIDAWGHGTEVIVSS | | | | | | | |
| H06_R2 | AVTLDESGGGLQTPGGALSLVCKAS | SEQ ID NO: 39 | GFTFSDHA | SEQ ID NO: 79 | ISSGTGSWT | SEQ ID NO: 119 | AKGAGSACCYAGSIDA | SEQ ID NO: 159 |
| | GFTFSDHAIFWVRQAPDKGLEFVAA | | | | | | | |
| | ISSGTGSWTAYATVVKGRATISRDN | | | | | | | |
| | GQSTVRLQLNNLRAADTATYYCAKG | | | | | | | |
| | AGSACCYAGSIDAWGHGTEVIVSS | | | | | | | |
| H07_R2_2 | AVTLDESGGGLQTPGGALSLVCKAS | SEQ ID NO: 40 | GFSISGYN | SEQ ID NO: 80 | ISTAGSYT | SEQ ID NO: 120 | AKHSGPGWYFGRGASAGL | SEQ ID NO: 160 |
| | GFSISGYNMAWVRQAPGKGLEWV | | | | | | | |
| | GAISTAGSYTAYAPAVKGRATISRDN | | | | | | | |
| | GQSTVRLQLNNLRAEDTGTYYCAKH | | | | | | | |
| | SGPGWYFGRGASAGLIDAWGHGTE | | | | | | | |
| | VIVSS | | | | | | | |
| R14_54 | AVTLDESGGGLQTPGGALSLVCKAS | SEQ ID NO 337 | GFKFSDHA | SEQ ID NO:338 | ISQGSGSWT | SEQ ID NO:339 | AKGAGRACCYAGSIDA | SEQ ID NO:340 |
| | GFKFSDHAIFWVRQAPDKGLEFVAA | | | | | | | |
| | ISQGSGSWTAYATVVKGRATISRDN | | | | | | | |
| | GQSTVRLQLNNLRAADTATYYCAKG | | | | | | | |
| | AGRACCYAGSIDAWGHGTEVIVSS | | | | | | | |
| ANTIBODY | | | | | | | | |
| NAME | VL SEQUENCE | | L-CDR1 | | L-CDR2 | | L-CDR3 | |
| 1G2_9 | ALTQPSSVSANPGETVKITCSGSSGS | SEQ ID NO: 161 | SGS | SEQ ID NO: 201 | WDD | SEQ ID NO: 241 | GSIDSSVGYVGI | SEQ ID NO: 281 |
| | YGWYQQKSPGSAPVTVIYWDDERP | | | | | | | |
| | SGIPSRFSGSASGSTATLTITGVQAED | | | | | | | |
| | EAVYFCGSIDSSVGYVGIFGAGTTLTV | | | | | | | |
| | L | | | | | | | |
| 2A2_12 | ALTQPSSVSANPGETVKITCSGGYSY | SEQ ID NO: 162 | YSYYGTYY | SEQ ID NO: 202 | RND | SEQ ID NO: 242 | GSYDSSDSGI | SEQ ID NO: 282 |
| | YGTYYYSWYQQKSPGSAPVTLIYRN | | | | | | | |
| | DKRPSDIPSRFSGSGSGSTSTLTITGV | | | | | | | |
| | QAEDEAVYFCGSYDSSDSGIFGAGTT | | | | | | | |
| | LTVL | | | | | | | |
| 2E5_22 | ALTQPSSVSANLGGTVKITCSGGSGN | SEQ ID NO: 163 | SGN | SEQ ID NO: 203 | SND | SEQ ID NO: 243 | GSRDSSYVGL | SEQ ID NO: 283 |
| | YGWYQQKSPGSVPVTVIYSNDKRPS | | | | | | | |
| | DIPSRFSGSGSGSTATLTITGVQVEDE | | | | | | | |
| | AVYFCGSRDSSYVGLFGAGTTLTVL | | | | | | | |
| 5G5_30 | ALTQPSSVSANPGETVKITCSGGSGS | SEQ ID NO: 164 | SGSSY | SEQ ID NO: 204 | YDD | SEQ ID NO: 244 | GTGDGSIAI | SEQ ID NO: 284 |
| | SYYGWYQQKSPGSAPVTVIYYDDKR | | | | | | | |
| | PSDIPSRFSGSKSGSTATLTITGVQVE | | | | | | | |
| | DEAVYYCGTGDGSIAIFGAGTTLTVL | | | | | | | |
| 6F12_33 | ALTQPSSVSANPGETVKITCSGGSSS | SEQ ID NO: 165 | SSSSY | SEQ ID NO: 205 | YND | SEQ ID NO: 245 | GTGDGSFAI | SEQID NO: 285 |
| | SYYGWYQQKSPGSAPVTLIYYNDKR | | | | | | | |
| | PSDIPSRFSGSKSGSTATLTITGVQVE | | | | | | | |
| | DEAVYYCGTGDGSFAIFGAGTTLTVL | | | | | | | |
| 7C3_39 | ALTQPSSVSANLGGTVKITCSGGGSS | SEQ ID NO: 166 | IGSSSY | SEQ ID NO: 206 | GND | SEQ ID NO: 246 | GSADSSDSGSGI | SEQ ID NO: 286 |
| | SYYGWYQQKSPGSAPVTVIYGNDRR | | | | | | | |
| | PSDIPSRFSGSASGSTATLTITGVQAE | | | | | | | |
| | DEAVYFCGSADSSDSGSGIFGAGTTL | | | | | | | |
| | TVL | | | | | | | |
| 51 | ALTQPSSVSANPGGTVKITCAGGGS | SEQ ID NO: | GSW | SEQ ID NO: 207 | QNN | SEQ ID NO: 247 | GSYDSSAGYAGGI | SEQID NO: 287 |
| | WYGWYQQKSPGSAPVSVIYQNNQ | 167 | | | | | | |
| | RPSDIPSRFSGSKSGSANTLTITGVQV | | | | | | | |
| | DDEAVYFCGSYDSSAGYAGGIFGAG | | | | | | | |
| | TTLTVL | | | | | | | |
| MD1_1A10 | ALTQPSSVSANPGETVKIICSGSSSSY | SEQ ID NO: 168 | SSS | SEQ ID NO: 208 | YND | SEQ ID NO: 248 | AGYDSSSYVGI | SEQ ID NO: 288 |
| | GWYQQKSPGSAPVTLIYYNDNRPSD | | | | | | | |
| | IPSRFSGSKSGSTGTLTITGVQADDEA | | | | | | | |
| | VYFCAGYDSSSYVGIFGAGTTLTVL | | | | | | | |
| MD1_1B6 | ALTQPSSVSANPGETVKITCSGGTSG | SEQ ID NO: 169 | TSG | SEQ ID NO: 209 | ERS | SEQ ID NO: 249 | GSRDSTTI | SEQ ID NO: 289 |
| | YAYGWYQQKSPGSALVTVIYERSKR | | | | | | | |
| | PSDIPSRFSGSTSGSTNTLTITGVQAD | | | | | | | |
| | DEAVYFCGSRDSTTIFGAGTTLTVL | | | | | | | |
| MD1_1C2 | ALTQPSSVSANPGETVKITCSGGGSS | SEQ ID NO: 170 | GSSY | SEQ ID NO: 210 | YND | SEQ ID NO: 250 | GTGDGSIAI | SEQID NO: 290 |
| | YYGWYQQKSPGSAPVTLIYYNDKRP | | | | | | | |
| | SDIPSRFSGSKSGSTATLTITGVQVED | | | | | | | |
| | EAVYYCGTGDGSIAIFGAGTTLTVL | | | | | | | |
| MD1_1D8 | ALTQPSSVSANPGQTVEITCSGSSGS | SEQ ID NO: | SGS | SEQ ID NO: 211 | NSN | SEQ ID NO: 251 | GSYEGIISFVGI | SEQ ID NO: 291 |
| | YGWHQQKSPGSAPVTVIYNSNQRP | 171 | | | | | | |
| | SDIPSRFSGSKSGSTATLTITGVQAED | | | | | | | |
| | EAVYFCGSYEGIISFVGIFGAGTTLTVL | | | | | | | |
| MD1_1F8 | ALTQPSSVSANPGETVKIICSGSSSSY | SEQ ID NO: 172 | SSS | SEQ ID NO: 212 | YND | SEQ ID NO: 252 | AGYDSSSYVGI | SEQ ID NO: 292 |
| | GWYQQKSPGSAPVTLIYYNDNRPSD | | | | | | | |
| | IPSRFSGSKSGSTGTLTIIGVQADDEA | | | | | | | |
| | VYFCAGYDSSSYVGIFGAGTTLTVL | | | | | | | |
| MD1_2A2 | ALTQPSSVSANLGGTVKITCSGGDS | SEQ ID NO: 173 | DSWYGSYY | SEQ ID NO: 213 | TND | SEQ ID NO: 253 | GAWDSISDVGI | SEQ ID NO: 293 |
| | WYGSYYYGWYQQKSPGSAPVTVIYT | | | | | | | |
| | NDKRPSDIPSRFSGSTSGSMATLTIT | | | | | | | |
| | GVRAEDEAVYFCGAWDSISDVGIFG | | | | | | | |
| | AGTTLTVL | | | | | | | |
| MD1_2D10 | ALTQPSSVSANPGETVKITCSGGGGS | SEQ ID NO: 174 | GGSSY | SEQ ID NO: 214 | YND | SEQ ID NO: 254 | ATGDGTYAV | SEQ ID NO: 294 |
| | SYYGWYQQKAPGSAPVTVIYYNDKR | | | | | | | |
| | PSDIPSRFSGSKFGSTATLTITGVQVE | | | | | | | |
| | DEAVYYCATGDGTYAVFGAGTTLTV | | | | | | | |
| | L | | | | | | | |
| MD1_2F9 | ALTQPSSVSANLGGTVEITCSGGGSY | SEQ ID NO: 175 | GSY | SEQ ID NO: 215 | SND | SEQ ID NO: 255 | GGYDRSNYNSI | SEQ ID NO: 295 |
| | AYGWYQQKSPGSAPVSLIYSNDKRP | | | | | | | |
| | SDIPSRFSGSKSGSTGTLTITGVQAED | | | | | | | |
| | EAVYFCGGYDRSNYNSIFGAGTTLTV | | | | | | | |
| | L | | | | | | | |
| S2K_C1 | ALTQPSSVSANPGETVKITCSGGSYY | SEQ ID NO: 176 | SYY | SEQ ID NO: 216 | YND | SEQ ID NO: 256 | GSFDSSVTGI | SEQ ID NO: 296 |
| | YQTYSYGWYQQKSPGSALVTVIYYN | | | | | | | |
| | DKRPSDIPSRFSGSKSGSTATLTITGV | | | | | | | |
| | QADDEAVYFCGSFDSSVTGIFGAGTT | | | | | | | |
| | LTVL | | | | | | | |
| S2K_H11 | ALTQPSSVSANPGETVKITCSGGGSS | SEQ ID NO: 177 | GSSSY | SEQ ID NO: 217 | QND | SEQ ID NO: 257 | GGYDSSAGI | SEQ ID NO: 297 |
| | SYYGWFQQKSPGSAPVTVIYQNDKR | | | | | | | |
| | PSDIPSRFSGSASGSTATLTITGVQVE | | | | | | | |
| | DEAVYFCGGYDSSAGIFGAGTTLTVL | | | | | | | |
| L12-1H - | ALAQPSSVSANPGETVKITCSGGSSS | SEQ ID NO: | SSSSY | SEQ ID NO: 218 | YND | SEQ ID NO: 258 | GTGDGSFAI | SEQ ID NO: 298 |
| L12-1L | SYYGWYQQKSPGSAPVTLIYYNDKR | 178 | | | | | | |
| | PSDIPSRFSGSKSGSTATLTITGVQVE | | | | | | | |
| | DEAVYYCGTGDGSFAIFGAGTTLTVL | | | | | | | |
| E01_R2 | ALTQPSSVSANPGETVEITCSGGTSG | SEQ ID NO: | TSG | SEQ ID NO: 219 | ERS | SEQ ID NO: 259 | GGRDSTEAI | SEQ ID NO: 299 |
| | YAYGWYQQKSPGSALVTVIYERSKR | 179 | | | | | | |
| | PSDIPSRFSGSTSGSTNTLTITGVQAD | | | | | | | |
| | DEAVYFCGGRDSTEAIFGAGTTLTVL | | | | | | | |
| E01_R3 | ALTQPSSVSANPGETVKIICSGSSSSY | SEQ ID NO: 180 | Isss | SEQ ID NO: 220 | YND | SEQ ID NO: 260 | GTGDGSIAI | SEQ ID NO: 300 |
| | GWYQQKSPGSAPVTLIYYNDKRPSD | | | | | | | |
| | IPSRFSGSKSGSTATLTITGVQVEDEA | | | | | | | |
| | VYYCGTGDGSIAIFGAGTTLTVL | | | | | | | |
| E02_R2_2 | ALTQPSSVSANPGETVKITCSGSSGS | SEQ ID NO: 181 | ISGS | SEQ ID NO: 221 | NGN | SEQ ID NO: 261 | GSYEGSTDTGI | SEQ ID NO: 301 |
| | YGWFQQKSPGSAPVTVIYNGNNRP | | | | | | | |
| | SDIPSRFSGSKSGSMGTLTITGVQAD | | | | | | | |
| | DEAVYFCGSYEGSTDTGIFGAGTTLT | | | | | | | |
| | VL | | | | | | | |
| E03_R3 | ALTQPSSVSANPGETVKITCSGSSGS | SEQ ID NO: | SGS | SEQ ID NO: 222 | NGN | SEQ ID NO: 262 | GSYEGSTDTGI | SEQID NO: 302 |
| | YGWFQQKSPGSAPVTVIYNGNNRP | 182 | | | | | | |
| | SDIPSRFSGSKSGSMGTLTITGVQAD | | | | | | | |
| | DEAVYFCGSYEGSTDTGIFGAGTTLT | | | | | | | |
| | VL | | | | | | | |
| E05_R2_2 | ALTQPSSVSANPGETVKITCSGGYSA | SEQID NO: 183 | YSA | SEQ ID NO: 223 | ERN | SEQ ID NO: 263 | GSTDSSTTI | SEQ ID NO: 303 |
| | YAYGWHQQKSPGSALVTVIYERNKR | | | | | | | |
| | PSDIPSRFSGSTSGSTGTLTITGVQAD | | | | | | | |
| | DEAVYYCGSTDSSTTIFGAGTTLTVL | | | | | | | |
| E06_R2 | ALTQPSSVSANPGETVKITCSGGYSD | SEQID NO: | YSDY | SEQ ID NO: 224 | ERR | SEQ ID NO: 264 | GGHDSSGSTI | SEQ ID NO: 304 |
| | YAYGWYQQKSPGSAPLTVIYERRKR | 184 | | | | | | |
| | PSSIPSRFSGSTSGSTGTLTITGVQAD | | | | | | | |
| | DEAVYFCGGHDSSGSTIFGAGTTLTV | | | | | | | |
| | L | | | | | | | |
| E06_R2_2 | ALTQPSSVSANPGETVKITCSGSSGS | SEQ ID NO: 185 | SGS | SEQ ID NO: 225 | NGN | SEQ ID NO: 265 | GSYEGSTDTGI | SEQ ID NO: 305 |
| | YGWFQQKSPGSAPVTVIYNGNNRP | | | | | | | |
| | SDIPSRFSGSKSGSMGTLTITGVQAD | | | | | | | |
| | DEAVYFCGSYEGSTDTGIFGAGTTLT | | | | | | | |
| | VL | | | | | | | |
| E07_R2 | ALTQPSSVSSNLGGTVEITCSGGGPK | SEQ ID NO: | GPKSY | SEQ ID NO: 226 | AND | SEQ ID NO: 266 | GDIDTSGG | SEQID NO: 306 |
| | SYYGWYQQKSPGSAPVTVIYANDKR | 186 | | | | | | |
| | PSDIPSRFSGSKSGSTHTLTITGVQAE | | | | | | | |
| | DEAVYFCGDIDTSGGFGAGTTLTVL | | | | | | | |
| E08_R2_2 | ALTQPSSVSANPGETVKITCSGSSGS | SEQ ID NO: 187 | SGS | SEQ ID NO: 227 | NGN | SEQ ID NO: 267 | GSYEGSTDTGI | SEQ ID NO: 307 |
| | YGWFQQKSPGSAPVTVIYNGNNRP | | | | | | | |
| | SDIPSRFSGSKSGSMGTLTITGVQAD | | | | | | | |
| | DEAVYFCGSYEGSTDTGIFGAGTTLT | | | | | | | |
| | VL | | | | | | | |
| F02_R2_2 | ALTQPSSVSANPGETVKITCSGSSGS | SEQ ID NO: 188 | SGS | SEQ ID NO: 228 | NGN | SEQ ID NO: 268 | GSYEGSTDTGI | SEQ ID NO: 308 |
| | YGWFQQKSPGSAPVTVIYNGNNRP | | | | | | | |
| | SDIPSRFSGSKSGSMGTLTITGVQAD | | | | | | | |
| | DEAVYFCGSYEGSTDTGIFGAGTTLT | | | | | | | |
| | VL | | | | | | | |
| F05_R2 | ALTQPSSASANPGETVKITCSGGYSG | SEQ ID NO: 189 | YSGY | SEQ ID NO: 229 | ERS | SEQ ID NO: 269 | GGHDSSSI | SEQ ID NO: 309 |
| | YAYGWYQQKSPGSALVTVIYERSKR | | | | | | | |
| | PSDIPSRFSGSTSDSTNTLTITGVQAD | | | | | | | |
| | DEAVYFCGGHDSSSIFGAGTTLTVL | | | | | | | |
| F06_R2 | ALTQPSSVSANPGETVKITCSGGYSD | SEQ ID NO: 190 | YSDY | SEQ ID NO: 230 | ERN | SEQ ID NO: 270 | GGHDSSSI | SEQ ID NO: 310 |
| | YAYGWYQQKSPGSALVTVIYERNKR | | | | | | | |
| | PSNIPSRFSGSRSGSTNTLTITGVQAD | | | | | | | |
| | DEAVYFCGGHDSSSIFGAGTTLTVL | | | | | | | |
| G06_R3 | ALTQPSSVSANPGETVKITCSGGRYF | SEQ ID NO: 191 | RY | SEQ ID NO: 231 | DDT | SEQ ID NO: 271 | GGYDSSTYAGI | SEQ ID NO: 311 |
| | GWFQQKAPGSAPVTLIYDDTNRPSN | | | | | | | |
| | IPSRFSGSTSGSTSTLTITGVRAEDEA | | | | | | | |
| | VYYCGGYDSSTYAGIFGAGTTLTVL | | | | | | | |
| G07_R2 | ALTQPSSVSANPGETVKITCSGGSSS | SEQ ID NO: 192 | SSSSY | SEQ ID NO: 232 | YND | SEQ ID NO: 272 | GTGDGSFAI | SEQ ID NO: 312 |
| | SYYGWYQQKSPGSAPVTLIYYNDKR | | | | | | | |
| | PSDIPSRFSGSKSGSTATLTITGVQVE | | | | | | | |
| | DEAVYYCGTGDGSFAIFGAGTTLTVL | | | | | | | |
| G09_R2 | ALTQPSSVSANPGETVKITCSGGSSS | SEQ ID NO: 193 | SSSSY | SEQ ID NO: 233 | YND | SEQ ID NO: 273 | GTGDGSFAI | SEQ ID NO: 313 |
| | SYYGWYQQKSPGSAPVTLIYYNDKR | | | | | | | |
| | PSDIPSRFSGSKSGSTATLTITGVQVE | | | | | | | |
| | DEAVYYCGTGDGSFAIFGAGTTLTVL | | | | | | | |
| G10_R2 | ALTQPSSVSANPGETVKITCSGGSSS | SEQ ID NO: 194 | SSSSY | SEQ ID NO: 234 | YND | SEQ ID NO: 274 | GTGDGSFAI | SEQ ID NO: 314 |
| | SYYGWYQQKSPGSAPVTLIYYNDKR | | | | | | | |
| | PSDIPSRFSGSKSGSTATLTITGVQVE | | | | | | | |
| | DEAVYYCGTGDGSFAIFGAGTTLTVL | | | | | | | |
| H01_R2 | ALTQPSSVSANPGETVKITCSGGSSS | SEQ ID NO: 195 | SSSSY | SEQ ID NO: 235 | YND | SEQ ID NO: 275 | GSADSSDSGSGI | SEQ ID NO: 315 |
| | SYYGWYQQKSPGSAPVTLIYYNDKR | | | | | | | |
| | PSDIPSRFSGSKSGSTATLTITGVQVE | | | | | | | |
| | DEAVYYCGSADSSDSGSGIFGAGTTL | | | | | | | |
| | TVL | | | | | | | |
| H02_R2 | ALTQPSSVSANPGETVKITCSGGGSS | SEQ ID NO: 196 | GSS | SEQ ID NO: 236 | GND | SEQ ID NO: 276 | GSADSSDSGSGI | SEQ ID NO: 316 |
| | YGWYQQKSPGSAPVTVIYGNDRRPS | | | | | | | |
| | DIPSRFSGSASGSTATLTITGVQAEDE | | | | | | | |
| | AVYFCGSADSSDSGSGIFGAGTTLTV | | | | | | | |
| | L | | | | | | | |
| H03_R3 | ALTQPSSVSANPGETVKITCSGGSSS | SEQ ID NO: 197 | SSSSY | SEQ ID NO: 237 | YND | SEQ ID NO: 277 | GTGDGSFAI | SEQ ID NO: 317 |
| | SYYGWYQQKSPGSAPVTLIYYNDKR | | | | | | | |
| | PSDIPSRFSGSKSGSTATLTITGVQVE | | | | | | | |
| | DEAVYYCGTGDGSFAIFGAGTTLTVL | | | | | | | |
| H05_R2_2 | ALTQPSSVSANPGETVKITCSGGSSS | SEQ ID NO: 198 | SSSSY | SEQ ID NO: 238 | YND | SEQ ID NO: 278 | GTGDGSFAI | SEQ ID NO: 318 |
| | SYYGWYQQKSPGSAPVTLIYYNDKR | | | | | | | |
| | PSDIPSRFSGSKSGSTATLTITGVQVE | | | | | | | |
| | DEAVYYCGTGDGSFAIFGAGTTLTVL | | | | | | | |
| H06_R2 | ALTQPSSVSANLGGTVKITCSGGSSS | SEQ ID NO: | SSSSY | SEQ ID NO: 239 | YND | SEQ ID NO: 279 | GTGDGSFAI | SEQ ID NO: 319 |
| | SYYGWYQQKSPGSAPVTLIYYNDKR | 199 | | | | | | |
| | PSDIPSRFSGSKSGSTATLTITGVQVE | | | | | | | |
| | DEAVYYCGTGDGSFAIFGAGTTLTVL | | | | | | | |
| H07_R2_2 | ALTQPSSVSANPGETVKITCSGGSGS | SEQ ID NO: 200 | SGS | SEQ ID NO: 240 | DNT | SEQ ID NO: 280 | GAYDSSSYIGI | SEQ ID NO: 320 |
| | YGWYQQKTPGSAPVTVIYDNTNRPS | | | | | | | |
| | NIPSRFSGSTSGSTGTLTITGVQVEDE | | | | | | | |
| | AVYYCGAYDSSSYIGIFGAGTTLTVL | | | | | | | |
| R14_54 | ALTQPSSVSANPGETVKITCSGGSSS | SEQ ID NO:341 | SSSY | SEQ ID NO:342 | YND | SEQ ID NO:343 | GTGDGSFAI | SEQ ID NO:344 |
| | SYYGWYQQKSPGSAPVTLIYYNDKR | | | | | | | |
| | PSDIPSRFSGSKSGSTATLTITGVQVE | | | | | | | |
| | DEAVYYCGTGDGSFAIFGAGTTLTVL | | | | | | | |

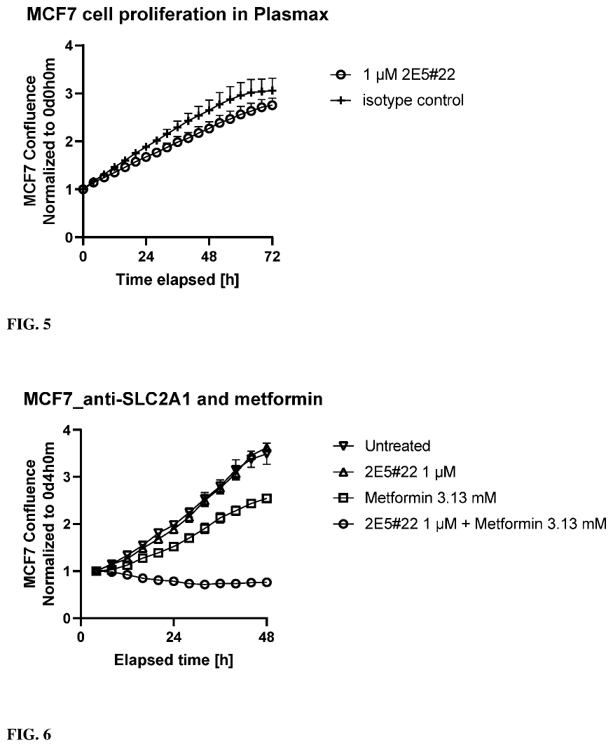

ELISA results indicate the anti-SLC2A1 antibodies bind their target expressed on VLP surface with high affinity (FIG. 1). Furthermore, the antibodies bind to HepG2 tumor cells that express SLC2A1 endogenously (FIG. 2). The antibodies are highly specific for SLC2A1 as they do not bind to HepG2 cells that are negative for SLC2A1 (knock out (KO) HepG2 cells, Abcam, ab280797). The specific binding to SLC2A1 in comparison to other related glucose transporters was demonstrated in an ELISA-based SLC2A2, SLC2A3 and SLC2A4 VLP binding assay (FIG. 3). Some binding of 2E5 #22 antibody was observed to other VLPs than SLC2A1 which is due to low endogenous levels of SLC2A1 on all VLPs (determined by mass spectrometry, data not shown). Importantly, the anti-SLC2A1 antibodies described here are potent inhibitors of glucose uptake (FIG. 4). Glucose uptake inhibition directly influences the ability of cancer cell to fuel its growth via glycolysis. However, this might force the cells to rely on OXPHOS as an alternative to sustain proliferation. Indeed, when MCF7 breast cancer cells were incubated in the presence of anti-SLC2A1 antibody 2E5 #22, only a modest inhibition of cell growth was observed using Incucyte Live-Cell Analysis system which tracks cell growth as changes in confluence or cell count over time (FIG. 5). Metformin, phenformin and IACS-010759 are all inhibitors of OXPHOS. We used anti-SLC2A1 antibodies in combination with metformin, phenformin and IACS-010759 to inhibit both glycolysis and OXPHOS simultaneously and enhance the growth inhibition. When MCF7 breast cancer cells were treated with anti-SLC2A1 5G5 #30 or 6F12 #33 in combination with metformin, phenformin or IACS-010759, MCF7 breast cancer cell growth was drastically inhibited (FIGS. 6, 7 and 8 respectively). Metformin, phenformin or IACS-010759 and anti-SLC2A1 combination treatment effects were much greater than growth inhibition induced by anti-SLC2A1 alone (FIGS. 6, 7 and 8). Metformin, phenformin or IACS-010759 alone, however, did not influence cell growth of the MCF7 breast cancer cell line (FIGS. 6, 7 and 8, respectively).

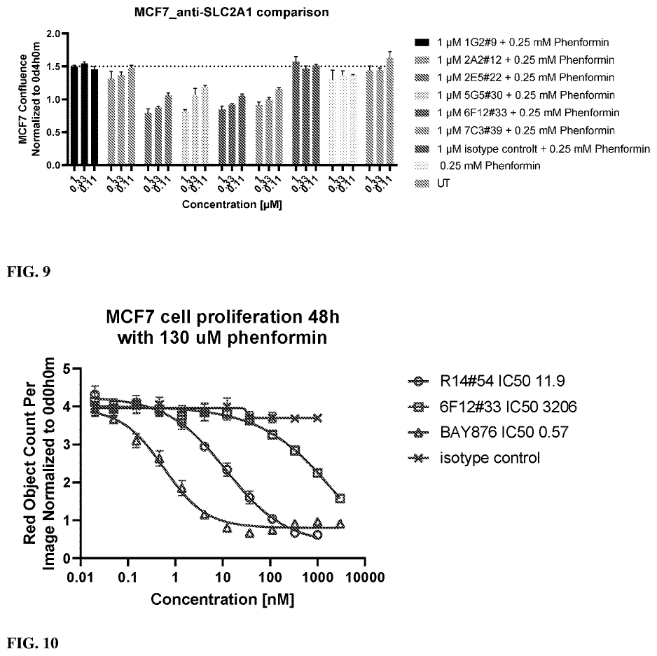

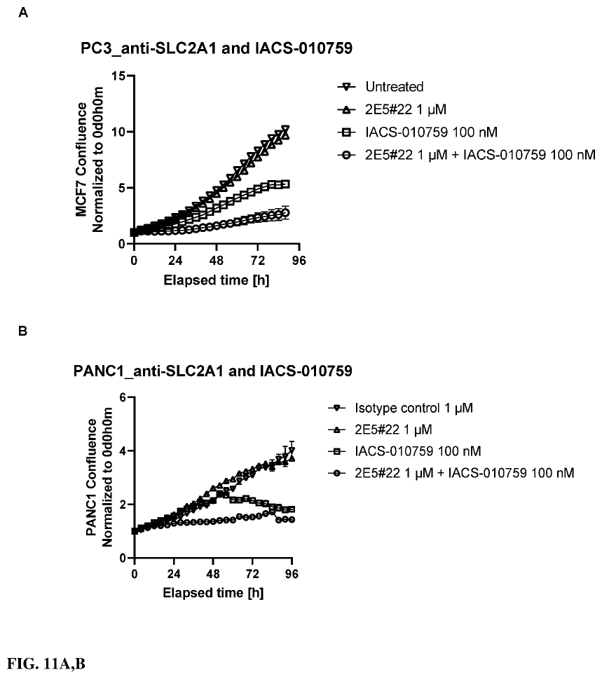

The fast potent anti-proliferative effect of anti-SLC2A1 antibody in combination OXPHOS inhibitors, such as phenformin, enables to compare anti-SLC2A1 antibodies easily after 24 hours of co-treatment incubation (FIG. 9). In FIG. 9, antibodies 2E5 #22, 5G5 #30, 6F12 #33 and 7C3 #39 stand out as most potent growth inhibitors of MCF7 breast cancer cell growth in combination with phenformin. No growth inhibition was detected when phenformin was used alone or combined with antibodies 1G2 #9 and 2A2 #12 or isotype control (FIG. 9). In FIG. 10, R14 #54 and 6F12 #33 anti-SLC2A1 antibodies demonstrate a concentration dependent growth inhibition of MCF7 cells in the presence of 130 μM phenformin and R14 #54 seems to be the most potent antibody.

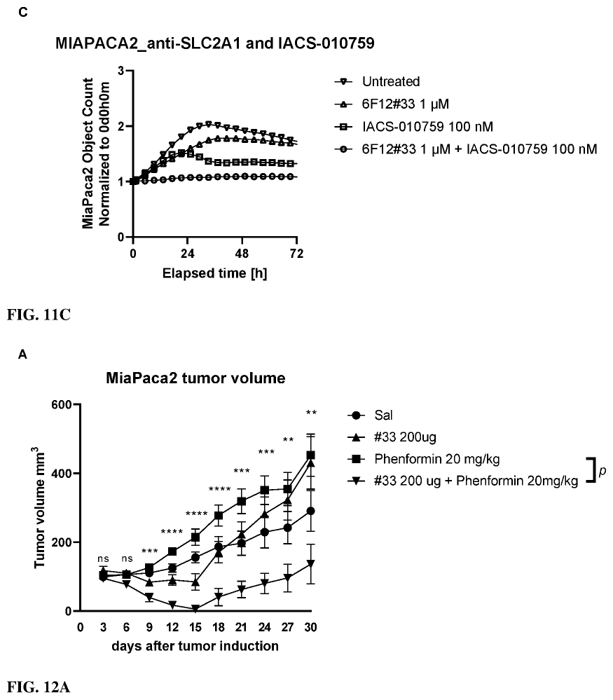

2E5 #22 and 6F12 #33 anti-SLC2A1 antibodies and OXPHOS inhibitor IACS-010759 were used as exemplary compounds to evaluate the effect of the combination therapy in other tumor types, and other compounds having similar OXPHOS inhibition effect may as well be used. A strong synergistic growth inhibition with 2E5 #22 and IACS-010759 was observed in prostate cancer cell line PC3 in comparison to single agent treatments (FIG. 11A). Furthermore, in pancreatic cancer cell lines PANC1 and MIAPACA2, the same dramatic growth reduction was detected with 6F12 #33 and IACS-010759 co-treatment in comparison to single agent treatments (FIGS. 11B and C, respectively). PC3 and MIAPACA2 cell growth is inhibited by IACS-010759 treatment alone whereas MIAPACA2 cells demonstrate sensitivity to anti-SCL2A1 single treatment (FIG. 11). This highlights the heterogeneity in metabolic pathways exploited by the tumor cells. Nonetheless, combining anti-SCL2A1 antibody with the OXPHOS inhibitor has a superior growth inhibition effect and the in vitro results represented in FIG. 11 indicate that this combination could be an option to restrict the growth of a variety of tumor types.

In an in vivo experimental system, where immunocompromised mice were xenotransplanted with MIAPACA2 pancreatic cancer cells, anti-SLC2A1 antibodies alone did not have an impact on tumor growth. However, like observed in in vitro assays, anti-SLC2A1 antibody 6F12 #33 and phenformin cotreatment resulted in a statistically significant tumor growth inhibition (FIG. 12A). No changes in animal body mass were observed during the treatments (FIG. 12B) and the antibody levels were not decreasing (FIG. 12C).

### REFERENCES

[1] Yan, N. Trends Biochem. Sci. 38, 151-159 (2013).
[2] Jardetzky, O. Nat. 1966 2115052 211, 969-970 (1966).
[3] Quistgaard, E. M. et al. Nat. Struct. Mol. Biol. 2013 206 20, 766-768 (2013).
[4] Mueckler, M. et al. Mol. Aspects Med. 34, 121-138 (2013).
[5] Thorens, B. Diabetologia 58, 221-232 (2015).
[6] Leto, D. et al. Nat. Rev. Mol. Cell Biol. 13, 383-396 (2012).
[7] Vannucci, S. J. et al. Glia 21, 2-21 (1997).
[8] Moley, K. H. Semin. Reprod. Med. 17, 137-152 (1999).
[9] Hankir, M. K. et al. EMBO Rep. 19, e46404 (2018).
[10] Mueckler, M. et al. Science (80-.). 229, 941-945 (1985).
[11] Morgello, S. et al. Glia 14, 43-54 (1995).
[12] Shim, B. Y. et al. Int. J. Color. Dis. 2012 283 28, 375-383 (2012).
[13] Sun, H. W. et al. PLoS One 11, e0168907 (2016).
[14] Hanahan, D. et al. Cell 144, 646-674 (2011).
[15] Noguchi, Y. et al. Cancer Lett. 154, 175-182 (2000).
[16] Ito, S. et al. J. Natl. Cancer Inst. 94, 1080-1091 (2002).
[17] Seki, T. et al. Nature 608, 421-428 (2022).
[18] Wei, M. et al. Biochem. Biophys. Res. Commun. 503, 1154-1159 (2018).
[19] Wang, J. et al. Proc. Natl. Acad. Sci. U.S.A 116, 10744-10748 (2019).
[20] Lu, L. et al. J. Cell. Physiol. 228, 251-257 (2013).
[21] Warburg, O. et al. J. Gen. Physiol. 8, 519-530 (1927).
[22] Heiden, M. G. V. et al. Science (80-.). 324, 1029-1033 (2009).
[23] Yamamoto, T. et al. Biochem. Biophys. Res. Commun. 170, 223-230 (1990).
[24] Zhang, X. et al. BMC Cancer 21, 1-10 (2021).
[25] Beaney, R. P. Semin. Nucl. Med. 14, 324-341 (1984).
[26] Ojelabi, O. A. et al. J. Biol. Chem. 291, 26762 (2016).
[27] Siebeneicher, H. et al. ChemMedChem 11, 2261 (2016).
[28] Olszewski, K. et al. Cell Chem. Biol. 29, 423-435.e10 (2022).
[29] Koppula, P. et al. J. Biol. Chem. 292, 14240 (2017).
[30] Shin, C. S. et al. Nat. Commun. 2017 81 8, 1-11 (2017).
[31] Martínez-Reyes, I. et al. Nat. Commun. 2020111 11, 1-11 (2020).
[32] Yang, Y. et al. Cancer Genet. Cytogenet. 196, 45-55 (2010).
[33] Birsoy, K. et al. Nat. 2014 5087494 508, 108-112 (2014).
[34] Saxena, N. et al. JNCI J. Natl. Cancer Inst. 108, 287 (2016).
[35] Sottnik, J. L. et al. Clin. Exp. Metastasis 28, 865-875 (2011).

What is claimed is:

1. An antibody or antigen binding fragment thereof for binding SLC2A1 protein and inhibiting glucose uptake of a tumor cell.

2. The antibody or antigen binding fragment of claim 1, wherein the antibody or antigen binding fragment binds to SLC2A1 with EC50 values of less than 10 nM, more preferably less than 5 nM and most preferably less than 2 nM.

3. An antibody or antigen binding fragment of claim 1 or 2, wherein the antibody or antigen binding fragment comprises one or more complementary determining regions (CDRs) having amino acid sequence selected from the group consisting of:
a heavy chain CDR1 comprising amino acids selected from the group consisting of SEQ ID 41-80 and SEQ ID NO: 337, a heavy chain CDR2 comprising amino acids selected from the group consisting of SEQ ID 81-120 and SEQ ID NO: 339, a heavy chain CDR3 comprising amino acids selected from the group consisting of SEQ ID 121-160 and SEQ ID NO:340; and
a light chain CDR1 comprising amino acids selected from the group consisting of a SEQ ID NO: 201-240 and SEQ ID NO: 342; a light chain CDR2 comprising amino acids selected from the group consisting of SEQ ID NO 241-280 and SEQ ID NO:343; and a light chain CDR3 comprising amino acids selected from the group consisting according of SEQ ID NO: 281-320 and SEQ ID NO:344.

4. The antibody or antigen binding fragment according to any one of the previous claims comprising heavy chain variable region having an amino acid sequence according to anyone of SEQ ID NO:161-180 and SEQ ID NO:337 and/or light chain variable region having an amino acid sequence according to any one of SEQ ID NO: 1-40 and SEQ ID NO:341.

5. A method to inhibit glucose uptake of tumor cells, the method comprising a step of administering a compound comprising at least one anti-SLC2A1 antibody or antigen binding fragment thereof to a patient having a tumor.

6. The method of claim 5, wherein the antibody or antigen binding fragment is according to anyone of claims 1-4.

7. The method of claim 5 or 6, wherein the method comprises administering the compound simultaneously or in sequence with at least one inhibitor of oxidative phosphorylation.

8. The method of claim 7, wherein the at least one inhibitor of oxidative phosphorylation is selected from the group consisting of metformin, phenformin and IACS-010579.

9. A pharmaceutical composition for use in treating cancer, the composition comprising one or more anti-SLC2A1 antibodies or antigen binding fragment thereof optionally in combination with one or more inhibitors of oxidative phosphorylation.

10. The pharmaceutical composition of claim 9, wherein the one or more antibody or antigen binding fragment thereof is selected from antibodies and antigen binding fragments according to any one of claims 1-4.

11. The pharmaceutical composition of claim 9 or 10, wherein the one or more inhibitors of oxidative phosphorylation is selected from the group consisting of metformin, phenformin and IACS-010579.

12. The pharmaceutical composition of any of claims 9-11, wherein the composition inhibits metabolism of cancer cells selected from breast cancer cells, hepatocarcinoma cells, colorectal cancer cells, lung cancer cells, pancreatic cancer cells, biliary cancer cells, cervical cancer cells.

13. An anti-SLC2A1 antibody or a binding fragment thereof for use in treating an infectious disease.

14. A method to treat infectious disease wherein said method comprises administering an anti-SLC2A1 antibody or a binding fragment thereof to the patient.

15. The anti-SLC2A1 antibody or a binding fragment thereof of claim 13, or the method of claim 14, wherein the infectious disease is malaria.

16. An anti-SLC2A1 antibody or a binding fragment thereof for use in treating diabetes side effects.

17. A method to treat diabetes side effects in a patient, comprising administering an anti-SLC2A1 antibody or a binding fragment thereof to the patient.

18. A method to diagnose cancer, wherein the method comprises contacting a tissue from a patient suspected to have the cancer with an antiSLC2A1 antibody or fragment thereof and determining the presence of over-expression of SLC2A1 in the tissue as an indication of the presence of cancer, and wherein an over-expression is determined treating the patient.


SEQUENCE LISTING (344 sequences)

SEQ ID NO: 1 - protein, 130 aa, synthetic construct
AVTLDESGGGLQTPGGALSLVCKASGFTFSTKGMGWVRQTPGKGLEWVAGISSMRRYTYYAPAVKGRATISRDNGQSTVRLQLSSLRAEDSGTYYCARDATYCSWTTSTCGWSGGTIDAWGHGTEVIVSS

SEQ ID NO: 2 - protein, 130 aa, synthetic construct
AVTLDESGGGLQTPGGALSLVCKGSGFTFSTKGMMWVRQAPGKGLEWVAGISSMSRYTYYAPAVKGRATISRDNGQSAVRLQLSSLRAEDTGTYYCARDATYCSWTASTCGWSGGTIDAWGHGTEVIVSS

SEQ ID NO: 3 - protein, 130 aa, synthetic construct; identical to SEQ ID NO: 1

SEQ ID NO: 4 - protein, 124 aa, synthetic construct
AVTLDESGGGLQTPGGALSLVCKASGFTFSSHAIFWVRQAPDKGLEFVAAISSSSGSWTAYATVVKGRATISRDNGQSTVRLQLNNLRAEDTGTYYCAKAAGSACCYAGSIDAWGHGTEVIVSS

SEQ ID NO: 5 - protein, 124 aa, synthetic construct
AVTLDESGGGLQTPGGALSLVCKASGFTFSDHAIFWVRQAPDKGLEFVAAISSGTGSWTAYATVVKGRATISRDNGQSTVRLQLNNLRAADTATYYCAKGAGSACCYAGSIDAWGHGTEVIVSS

SEQ ID NO: 6 - protein, 130 aa, synthetic construct; identical to SEQ ID NO: 1

SEQ ID NO: 7 - protein, 128 aa, synthetic construct
AVTLDESGGGLQTPGGALSLVCKASGFSIRNYNMAWVRQAPGKGLEWVGAISTAGSYTSYGPAVKGRATISRDNGQSTVRLQLNNLRAEDTGTYYCAKHSGPGWYFGSGATAGLIDAWGHGTEVIVSS

SEQ ID NO: 8 - protein, 128 aa, synthetic construct
AVTLDESGGGLQTPGGALSLVCKASGFSIRNYNMAWVRQAPGKGLEWVGAISTAGSYTAYAPAVKGRATISRDNGQSTVRLQLNNLRAEDTGTYYCAKHSGPGWYFGRGASAGLIDAWGHGNEVIVSS

SEQ ID NO: 9 - protein, 125 aa, synthetic construct
AVTLDESGGGLQTPGGALSLVCKASGFTFSSYGMGWVRQAPGKGLEFVGGIRKDGSFTYYGAAVKGRATISRDNGQSTVRLQLNNLRAEDTGNYFCARGGGCSSCDNFAGFIDAWGHGTEVIVSS

SEQ ID NO: 10 - protein, 130 aa, synthetic construct; identical to SEQ ID NO: 1

SEQ ID NO: 11 - protein, 119 aa, synthetic construct
AVTLDEAGGGLQTPGGALSLVCKASGFTFSDRGIHWVRQAPGKGLEYVAGISSGSGSSTGYGAAVKGRATISRDNGQSTVRLQLNNLRAEDTGTYFCVRGACCSSIDAWGHGTEVIVSS

SEQ ID NO: 12 - protein, 128 aa, synthetic construct
AVTLDESGGGLQTPGGALSLVCKASGFSIRNYNMAWVRQAPGKGLEWVGAISTAGSYTAYAPAVKGRATISRDNGQSTVRLQLNNLRAEDTGTYYCAKHSGRGWYFGRGASAGLIDAWGHGNEVIVSS

SEQ ID NO: 13 - protein, 125 aa, synthetic construct
AVTLDESGGGLQTPGGALSLVCKASGFTFSSYGMGWVRQAPGKGLEFVAGIRKDGSYTYYGAAVKGRATISRDNGQSTLRLQLNSLRAEDTATYFCARGGGCNTCDNIAGFIDAWGHGTEVIVSS

SEQ ID NO: 14 - protein, 124 aa, synthetic construct
AVTLDESGGGLQTPGGGLSLVCKASGFTFSSYTMQWVRQAPGKGLEWVAGIANSPYYGAAVQGRATISRDNGQSTLRLQLNNLRAEDTATYYCAKSFDTYGCIYGRCAGRIDTWGHGTEVIVSS

SEQ ID NO: 15 - protein, 127 aa, synthetic construct
AVTLDESGGGLQTPGGGLSLVCKASGFSFSSHGMGWVRQAPGKGLEYVAAISGAGSWTGYGSAVKGRATISRDNGQSTMRLQLNNLRAEDTGTYYCAKSSFGCSNSCRNYAGTIDAWGHGTEVIVSS

SEQ ID NO: 16 - protein, 128 aa, synthetic construct
AVTLDESGGGLQTPGGALSLVCKASGFSISNYNMAWVRQAPGKGVEWVGAISTGGSYTAYGPAVKGRATISRDNGQSTMRLHLNNLRAEDTGTYYCAKHSEGGWYFGSGATAGLIDAWGHGTEVIVSS

SEQ ID NO: 17 - protein, 128 aa, synthetic construct
AVTLDESGGGLQTPGGALSLVCKASGFSISGYNMAWVRQAPGKGLEYVAGISTAGSYTGYAPAVKGRATISRDNGQSTVRLQLNNLRAEDTATYYCAKHSGPGWYFGRGSTAGRIDTWGHGTEVIVSS

SEQ ID NO: 18 - protein, 124 aa, synthetic construct
AATLDESGGGLQTPGGALSLVCKASGFTFSDHAIFWVRQAPDKGLEFVAAISSGTGSWTAYATVVKGRATISRDNGQSTVRLQLNNLRAADTATYYCAKGAGSACCYAGSIDAWGHGTEVIVSS

SEQ ID NO: 19 - protein, 125 aa, synthetic construct
AVTLDESGGGLQTPGGALSLVCKASGFTFSSYGMGWVRQAPGRGLEFVAGIRKDGSTTYYGAAVKGRATISRDNGQSTLRLQLNSLRAEDTATYFCARGGGCKTCDNIAGFIDAWGHGTEVIVSS

SEQ ID NO: 20 - protein, 128 aa, synthetic construct
AVTLDESGGGLQTPGGALSLVCKASGFSIRNYNMAWVRQAPGKGLEWVGAISTAGSYTAYAPAVKGRATISRDNGQSTVRLQLNNLRAEDTGTYYCAKHSGPGWYFGRGASAGLIDAWGHGTEVIVSS

SEQ ID NO: 21 - protein, 129 aa, synthetic construct
AVTLDESGGGLQTPGGALSVVCKASGLSIRNYNMAWVRQAPGKGLEWVGDISTAGIYTAYAPAVKGRATISRDNGQSTVRLQLNNLRAEDTGTYYCAKHCGPGWYFGSGGYVLGLIDAWGHGTEVIVSS

SEQ ID NO: 22 - protein, 128 aa, synthetic construct; identical to SEQ ID NO: 20

SEQ ID NO: 23 - protein, 125 aa, synthetic construct
AVTLDESGGGLQTPGGALSLVCKASGFTFSSYGMGWVRQAPGKGLEFVGAIRKDGSFTYYGAAVKGRATISRDNGQSTVRLQLSNLRAEDTGTYFCARGGGCSSCDNFAGFIDAWGHGTEVIVSS

SEQ ID NO: 24 - protein, 125 aa, synthetic construct
AVTLDESGGGLQTPGGALSLVCKASGFTFSSYGMGWVRQAPGKGLEFVAGIRNDGSYTYYGAAVKGRATISRDNGQSTLRLQLNSLRAEDTATYFCARGGGCNTCDNIAGFIDAWGHGTEVIVSS

SEQ ID NO: 25 - protein, 128 aa, synthetic construct
AVTLDESGGGLQTPGGALSLVCKASGFSIRNYNMAWVRQAPGKGLEWVGAISTAGSYTAYAPAVKGRATISRDNGQSTVRLQLNNLRDEDTGTYYCAKHSGPGWYFGRGASAGLIDAWGHGTEVIVSS

SEQ ID NO: 26 - protein, 125 aa, synthetic construct
AVTLDESGGGLQTPGGALSLVCKGSGFTFSSFHMFWVRQEPGKGLRWVAAISNDGSSAYGSAVKGRATISRDNGQGTVRLQLNNLRAEDTATYFCARSPGYCSGGRCYTTSIDAWGHGTEVIVSS

SEQ ID NO: 27 - protein, 127 aa, synthetic construct
AVTSDESGGGLQTPGGALSLVCKASGFSIRNYNMAWVRQAPGKGSEWVGTISTGGSYTAYAPAVKGRATISRDNGQSTVRLQLNNLRAEDTGTYCAKHSGPGWYFGRGASAGLIDAWGHGTEVIVSS

SEQ ID NO: 28 - protein, 127 aa, synthetic construct; identical to SEQ ID NO: 27

SEQ ID NO: 29 - protein, 125 aa, synthetic construct
AVTLDESGGGLQTPGGALSLVCKASGFTFSSYGMGWVRQAPGKGLEFVAAIRNDGSFTYYGAAVKGRATISRDNGQSTLRLQLNNLRTEDTGTYFCARGGGCSSCDNFAGFIDAWGHGTEVIVSS

SEQ ID NO: 30 - protein, 125 aa, synthetic construct
AVTLDESGGGLQTPGGALSLVCKASGFTFSSYGMGWVRQAPGKGLEFVAGIRKDGTYTYYGAAVKGRATISRDNGQSTLRLQLNNLRAEDTGTYFCARGGGCNTCDNIAGFIDAWGHGTEVIVSS

SEQ ID NO: 31 - protein, 128 aa, synthetic construct; identical to SEQ ID NO: 20

SEQ ID NO: 32 - protein, 128 aa, synthetic construct; identical to SEQ ID NO: 20

SEQ ID NO: 33 - protein, 124 aa, synthetic construct; identical to SEQ ID NO: 4

SEQ ID NO: 34 - protein, 128 aa, synthetic construct
AVTLDESGGGLQTPGGALSLVCKASGFSISGYNMAWVRQAPGKGLEWVGAISTAGSYTAYAPAVKGRATISRDNGQSTVRLQLNNLRAEDTGTYYCAKHSGPGWYFGRGASAGLIDAWGHGTEVIVSS

SEQ ID NO: 35 - protein, 128 aa, synthetic construct
AVTLDESGGGLQTPGGALSLVCKASGFSIRNYNMAWVRQAPGKGLEWVGAISTAGSYTAYAPAVKGRATITRDNGQSTVRLQLNNLRAEDTGTYYCAKHSGPGWYFGRGASAGLIDAWGHGTEVIVSS

SEQ ID NO: 36 - protein, 128 aa, synthetic construct; identical to SEQ ID NO: 20

SEQ ID NO: 37 - protein, 128 aa, synthetic construct
AVTLDESGGGLQTPGGGLSLVCKASGFSIRNYNMAWVRQAPGKGLEWVGAISTAGSYTAYAPAVKGRATISRDNGQSTVRLQLNNLRAEDTGTYYCAKHSGPGWYFGRGASAGLIDAWGHGTEVIVSS

SEQ ID NO: 38 - protein, 124 aa, synthetic construct
AVTLDESGGGLQTAGGALSLVCKASGFTFSDHAIFWVRQAPDKGLEFVAAISSGTGSWTAYATVVKGRATISRDNGQSTVRLQVNNLRAADTATYYCAKGAGSACCYAGSIDAWGHGTEVIVSS

SEQ ID NO: 39 - protein, 124 aa, synthetic construct; identical to SEQ ID NO: 5

SEQ ID NO: 40 - protein, 128 aa, synthetic construct; identical to SEQ ID NO: 34

SEQ ID NO: 41 - protein, 8 aa, synthetic construct
GFTFSTKG

SEQ ID NO: 42 - protein, 8 aa, synthetic construct
GFTFSTKG

SEQ ID NO: 43 - protein, 8 aa, synthetic construct
GFTFSTKG

SEQ ID NO: 44 - protein, 8 aa, synthetic construct
GFTFSSHA

SEQ ID NO: 45 - protein, 8 aa, synthetic construct
GFTFSDHA

SEQ ID NO: 46 - protein, 8 aa, synthetic construct
GFTFSTKG

SEQ ID NO: 47 - protein, 8 aa, synthetic construct
GFSIRNYN

SEQ ID NO: 48 - protein, 8 aa, synthetic construct
GFSIRNYN

SEQ ID NO: 49 - protein, 8 aa, synthetic construct
GFTFSSYG

SEQ ID NO: 50 - protein, 8 aa, synthetic construct
GFTFSTKG

SEQ ID NO: 51 - protein, 8 aa, synthetic construct
GFTFSDRG

SEQ ID NO: 52 - protein, 8 aa, synthetic construct
GFSIRNYN

SEQ ID NO: 53 - protein, 8 aa, synthetic construct
GFTFSSYG

SEQ ID NO: 54 - protein, 8 aa, synthetic construct
GFTFSSYT

SEQ ID NO: 55 - protein, 8 aa, synthetic construct
GFSFSSHG

SEQ ID NO: 56 - protein, 8 aa, synthetic construct
GFSISNYN

SEQ ID NO: 57 - protein, 8 aa, synthetic construct
GFSISGYN

SEQ ID NO: 58 - protein, 8 aa, synthetic construct
GFTFSDHA

SEQ ID NO: 59 - protein, 8 aa, synthetic construct
GFTFSSYG

SEQ ID NO: 60 - protein, 8 aa, synthetic construct
GFSIRNYN

SEQ ID NO: 61 - protein, 8 aa, synthetic construct
GLSIRNYN

SEQ ID NO: 62 - protein, 8 aa, synthetic construct
GFSIRNYN

SEQ ID NO: 63 - protein, 8 aa, synthetic construct
GFTFSSYG

SEQ ID NO: 64 - protein, 8 aa, synthetic construct
GFTFSSYG

SEQ ID NO: 65 - protein, 8 aa, synthetic construct
GFSIRNYN

SEQ ID NO: 66 - protein, 8 aa, synthetic construct
GFTFSSFH

SEQ ID NO: 67 - protein, 8 aa, synthetic construct
GFSIRNYN

SEQ ID NO: 68 - protein, 8 aa, synthetic construct
GFSIRNYN

SEQ ID NO: 69 - protein, 8 aa, synthetic construct
GFTFSSYG

SEQ ID NO: 70 - protein, 8 aa, synthetic construct
GFTFSSYG

SEQ ID NO: 71 - protein, 8 aa, synthetic construct
GFSIRNYN

SEQ ID NO: 72 - protein, 8 aa, synthetic construct
GFSIRNYN

SEQ ID NO: 73 - protein, 8 aa, synthetic construct
GFTFSSHA

SEQ ID NO: 74 - protein, 8 aa, synthetic construct
GFSISGYN

SEQ ID NO: 75 - protein, 8 aa, synthetic construct
GFSIRNYN

SEQ ID NO: 76 - protein, 8 aa, synthetic construct
GFSIRNYN

SEQ ID NO: 77 - protein, 8 aa, synthetic construct
GFSIRNYN

SEQ ID NO: 78 - protein, 8 aa, synthetic construct
GFTFSDHA

SEQ ID NO: 79 - protein, 8 aa, synthetic construct
GFTFSDHA

SEQ ID NO: 80 - protein, 8 aa, synthetic construct
GFSISGYN

SEQ ID NO: 81 - protein, 8 aa, synthetic construct
ISSMRRYT

SEQ ID NO: 82 - protein, 8 aa, synthetic construct
ISSMSRYT

SEQ ID NO: 83 - protein, 8 aa, synthetic construct
ISSMRRYT

SEQ ID NO: 84 - protein, 9 aa, synthetic construct
ISSSSGSWT

SEQ ID NO: 85 - protein, 9 aa, synthetic construct
ISSGTGSWT

SEQ ID NO: 86 - protein, 8 aa, synthetic construct
ISSMRRYT

SEQ ID NO: 87 - protein, 8 aa, synthetic construct
ISTAGSYT

SEQ ID NO: 88 - protein, 8 aa, synthetic construct
ISTAGSYT

SEQ ID NO: 89 - protein, 8 aa, synthetic construct
IRKDGSFT

SEQ ID NO: 90 - protein, 8 aa, synthetic construct
ISSMRRYT

SEQ ID NO: 91 - protein, 9 aa, synthetic construct
ISSGSGSST

SEQ ID NO: 92 - protein, 8 aa, synthetic construct
ISTAGSYT

SEQ ID NO: 93 - protein, 8 aa, synthetic construct
IRKDGSYT

SEQ ID NO: 94 - protein, 5 aa, synthetic construct
IANSP

SEQ ID NO: 95 - protein, 8 aa, synthetic construct
ISGAGSWT

SEQ ID NO: 96 - protein, 8 aa, synthetic construct
ISTGGSYT

SEQ ID NO: 97 - protein, 8 aa, synthetic construct
ISTAGSYT

SEQ ID NO: 98 - protein, 9 aa, synthetic construct
ISSGTGSWT

SEQ ID NO: 99 - protein, 8 aa, synthetic construct
IRKDGSTT

SEQ ID NO: 100 - protein, 8 aa, synthetic construct
ISTAGSYT

SEQ ID NO: 101 - protein, 8 aa, synthetic construct
ISTAGIYT

SEQ ID NO: 102 - protein, 8 aa, synthetic construct
ISTAGSYT

SEQ ID NO: 103 - protein, 8 aa, synthetic construct
IRKDGSFT

SEQ ID NO: 104 - protein, 8 aa, synthetic construct
IRNDGSYT

SEQ ID NO: 105 - protein, 8 aa, synthetic construct
ISTAGSYT

SEQ ID NO: 106 - protein, 7 aa, synthetic construct
ISNDGSS

SEQ ID NO: 107 - protein, 8 aa, synthetic construct
ISTAGSYT

SEQ ID NO: 108 - protein, 8 aa, synthetic construct
ISTGGSYT

SEQ ID NO: 109 - protein, 8 aa, synthetic construct
IRNDGSFT

SEQ ID NO: 110 - protein, 8 aa, synthetic construct
IRKDGTYT

SEQ ID NO: 111 - protein, 8 aa, synthetic construct
ISTAGSYT

SEQ ID NO: 112 - protein, 8 aa, synthetic construct
ISTAGSYT

SEQ ID NO: 113 - protein, 9 aa, synthetic construct
ISSSSGSWT

SEQ ID NO: 114 - protein, 8 aa, synthetic construct
ISTAGSYT

SEQ ID NO: 115 - protein, 8 aa, synthetic construct
ISTAGSYT

SEQ ID NO: 116 - protein, 8 aa, synthetic construct
ISTAGSYT

SEQ ID NO: 117 - protein, 8 aa, synthetic construct
ISTAGSYT

SEQ ID NO: 118 - protein, 9 aa, synthetic construct
ISSGTGSWT

SEQ ID NO: 119 - protein, 9 aa, synthetic construct
ISSGTGSWT

SEQ ID NO: 120 - protein, 8 aa, synthetic construct
ISTAGSYT

SEQ ID NO: 121 - protein, 23 aa, synthetic construct
ARDATYCSWTTSTCGWSGGTIDA

SEQ ID NO: 122 - protein, 23 aa, synthetic construct
ARDATYCSWTASTCGWSGGTIDA

SEQ ID NO: 123 - protein, 23 aa, synthetic construct
ARDATYCSWTTSTCGWSGGTIDA

SEQ ID NO: 124 - protein, 16 aa, synthetic construct
AKAAGSACCYAGSIDA

SEQ ID NO: 125 - protein, 16 aa, synthetic construct
AKGAGSACCYAGSIDA

SEQ ID NO: 126 - protein, 23 aa, synthetic construct
ARDATYCSWTTSTCGWSGGTIDA

SEQ ID NO: 127 - protein, 21 aa, synthetic construct
AKHSGPGWYFGSGATAGLIDA

SEQ ID NO: 128 - protein, 21 aa, synthetic construct
AKHSGPGWYFGRGASAGLIDA

SEQ ID NO: 129 - protein, 18 aa, synthetic construct
ARGGGCSSCDNFAGFIDA

SEQ ID NO: 130 - protein, 23 aa, synthetic construct
ARDATYCSWTTSTCGWSGGTIDA

SEQ ID NO: 131 - protein, 11 aa, synthetic construct
VRGACCSSIDA

SEQ ID NO: 132 - protein, 21 aa, synthetic construct
AKHSGRGWYFGRGASAGLIDA

SEQ ID NO: 133 - protein, 18 aa, synthetic construct
ARGGGCNTCDNIAGFIDA

SEQ ID NO: 134 - protein, 20 aa, synthetic construct
AKSFDTYGCIYGRCAGRIDT

SEQ ID NO: 135 - protein, 20 aa, synthetic construct
AKSSFGCSNSCRNYAGTIDA

SEQ ID NO: 136 - protein, 21 aa, synthetic construct
AKHSEGGWYFGSGATAGLIDA

SEQ ID NO: 137 - protein, 21 aa, synthetic construct
AKHSGPGWYFGRGSTAGRIDT

SEQ ID NO: 138 - protein, 16 aa, synthetic construct
AKGAGSACCYAGSIDA

SEQ ID NO: 139 - protein, 18 aa, synthetic construct
ARGGGCKTCDNIAGFIDA

SEQ ID NO: 140 - protein, 21 aa, synthetic construct
AKHSGPGWYFGRGASAGLIDA

SEQ ID NO: 141 - protein, 22 aa, synthetic construct
AKHCGPGWYFGSGGYVLGLIDA

SEQ ID NO: 142 - protein, 21 aa, synthetic construct
AKHSGPGWYFGRGASAGLIDA

SEQ ID NO: 143 - protein, 18 aa, synthetic construct
ARGGGCSSCDNFAGFIDA

SEQ ID NO: 144 - protein, 18 aa, synthetic construct
ARGGGCNTCDNIAGFIDA

SEQ ID NO: 145 - protein, 21 aa, synthetic construct
AKHSGPGWYFGRGASAGLIDA

SEQ ID NO: 146 - protein, 19 aa, synthetic construct
ARSPGYCSGGRCYTTSIDA

SEQ ID NO: 147 - protein, 21 aa, synthetic construct
AKHSGPGWYFGRGASAGLIDA

SEQ ID NO: 148 - protein, 22 aa, synthetic construct
AKHCGPGWYFGSGGYVLGLIDA

SEQ ID NO: 149 - protein, 18 aa, synthetic construct
ARGGGCSSCDNFAGFIDA

SEQ ID NO: 150 - protein, 18 aa, synthetic construct
ARGGGCNTCDNIAGFIDA

SEQ ID NO: 151 - protein, 21 aa, synthetic construct
AKHSGPGWYFGRGASAGLIDA

SEQ ID NO: 152 - protein, 21 aa, synthetic construct
AKHSGPGWYFGRGASAGLIDA

SEQ ID NO: 153 - protein, 16 aa, synthetic construct
AKAAGSACCYAGSIDA

SEQ ID NO: 154 - protein, 21 aa, synthetic construct
AKHSGPGWYFGRGASAGLIDA

SEQ ID NO: 155 - protein, 21 aa, synthetic construct
AKHSGPGWYFGRGASAGLIDA

SEQ ID NO: 156 - protein, 21 aa, synthetic construct
AKHSGPGWYFGRGASAGLIDA

SEQ ID NO: 157 - protein, 21 aa, synthetic construct
AKHSGPGWYFGRGASAGLIDA

SEQ ID NO: 158 - protein, 16 aa, synthetic construct
AKGAGSACCYAGSIDA

SEQ ID NO: 159 - protein, 16 aa, synthetic construct
AKGAGSACCYAGSIDA

SEQ ID NO: 160 - protein, 21 aa, synthetic construct
AKHSGPGWYFGRGASAGLIDA

SEQ ID NO: 161 - protein, 105 aa, synthetic construct
ALTQPSSVSANPGETVKITCSGSSGSYGWYQQKSPGSAPVTVIYWDDERPSGIPSRFSGSASGSTATLTITGVQAEDEAVYFCGSIDSSVGYVGIFGAGTTLTVL

SEQ ID NO: 162 - protein, 108 aa, synthetic construct
ALTQPSSVSANPGETVKITCSGGYSYYGTYYYSWYQQKSPGSAPVTLIYRNDKRPSDIPSRFSGSGSGSTSTLTITGVQAEDEAVYFCGSYDSSDSGIFGAGTTLTVL

SEQ ID NO: 163 - protein, 103 aa, synthetic construct
ALTQPSSVSANLGGTVKITCSGGSGNYGWYQQKSPGSVPVTVIYSNDKRPSDIPSRFSGSGSGSTATLTITGVQVEDEAVYFCGSRDSSYVGLFGAGTTLTVL

SEQ ID NO: 164 - protein, 104 aa, synthetic construct
ALTQPSSVSANPGETVKITCSGGSGSSYYGWYQQKSPGSAPVTVIYYDDKRPSDIPSRFSGSKSGSTATLTITGVQVEDEAVYYCGTGDGSIAIFGAGTTLTVL

SEQ ID NO: 165 - protein, 104 aa, synthetic construct
ALTQPSSVSANPGETVKITCSGGSSSSYYGWYQQKSPGSAPVTLIYYNDKRPSDIPSRFSGSKSGSTATLTITGVQVEDEAVYYCGTGDGSFAIFGAGTTLTVL

SEQ ID NO: 166 - protein, 107 aa, synthetic construct
ALTQPSSVSANLGGTVKITCSGGGSSSYYGWYQQKSPGSAPVTVIYGNDRRPSDIPSRFSGSASGSTATLTITGVQAEDEAVYFCGSADSSDSGSGIFGAGTTLTVL

SEQ ID NO: 167 - protein, 106 aa, synthetic construct
ALTQPSSVSANPGGTVKITCAGGGSWYGWYQQKSPGSAPVSVIYQNNQRPSDIPSRFSGSKSGSANTLTITGVQVDDEAVYFCGSYDSSAGYAGGIFGAGTTLTVL

SEQ ID NO: 168 - protein, 104 aa, synthetic construct
ALTQPSSVSANPGETVKIICSGSSSSYGWYQQKSPGSAPVTLIYYNDNRPSDIPSRFSGSKSGSTGTLTITGVQADDEAVYFCAGYDSSSYVGIFGAGTTLTVL

SEQ ID NO: 169 - protein, 103 aa, synthetic construct
ALTQPSSVSANPGETVKITCSGGTSGYAYGWYQQKSPGSALVTVIYERSKRPSDIPSRFSGSTSGSTNTLTITGVQADDEAVYFCGSRDSTTIFGAGTTLTVL

SEQ ID NO: 170 - protein, 103 aa, synthetic construct
ALTQPSSVSANPGETVKITCSGGGSSYYGWYQQKSPGSAPVTLIYYNDKRPSDIPSRFSGSKSGSTATLTITGVQVEDEAVYYCGTGDGSIAIFGAGTTLTVL

SEQ ID NO: 171 - protein, 105 aa, synthetic construct
ALTQPSSVSANPGQTVEITCSGSSGSYGWHQQKSPGSAPVTVIYNSNQRPSDIPSRFSGSKSGSTATLTITGVQAEDEAVYFCGSYEGIISFVGIFGAGTTLTVL

SEQ ID NO: 172 - protein, 104 aa, synthetic construct
ALTQPSSVSANPGETVKIICSGSSSSYGWYQQKSPGSAPVTLIYYNDNRPSDIPSRFSGSKSGSTGTLTIIGVQADDEAVYFCAGYDSSSYVGIFGAGTTLTVL

SEQ ID NO: 173 - protein, 109 aa, synthetic construct
ALTQPSSVSANLGGTVKITCSGGDSWYGSYYYGWYQQKSPGSAPVTVIYTNDKRPSDIPSRFSGSTSGSMATLTITGVRAEDEAVYFCGAWDSISDVGIFGAGTTLTVL

SEQ ID NO: 174 - protein, 104 aa, synthetic construct
ALTQPSSVSANPGETVKITCSGGGGSSYYGWYQQKAPGSAPVTVIYYNDKRPSDIPSRFSGSKFGSTATLTITGVQVEDEAVYYCATGDGTYAVFGAGTTLTVL

SEQ ID NO: 175 - protein, 105 aa, synthetic construct
ALTQPSSVSANLGGTVEITCSGGGSYAYGWYQQKSPGSAPVSLIYSNDKRPSDIPSRFSGSKSGSTGTLTITGVQAEDEAVYFCGGYDRSNYNSIFGAGTTLTVL

SEQ ID NO: 176 - protein, 108 aa, synthetic construct
ALTQPSSVSANPGETVKITCSGGSYYYQTYSYGWYQQKSPGSALVTVIYYNDKRPSDIPSRFSGSKSGSTATLTITGVQADDEAVYFCGSFDSSVTGIFGAGTTLTVL

SEQ ID NO: 177 - protein, 104 aa, synthetic construct
ALTQPSSVSANPGETVKITCSGGGSSSYYGWFQQKSPGSAPVTVIYQNDKRPSDIPSRFSGSASGSTATLTITGVQVEDEAVYFCGGYDSSAGIFGAGTTLTVL

SEQ ID NO: 178 - protein, 104 aa, synthetic construct
ALAQPSSVSANPGETVKITCSGGSSSSYYGWYQQKSPGSAPVTLIYYNDKRPSDIPSRFSGSKSGSTATLTITGVQVEDEAVYYCGTGDGSFAIFGAGTTLTVL

SEQ ID NO: 179 - protein, 104 aa, synthetic construct
ALTQPSSVSANPGETVEITCSGGTSGYAYGWYQQKSPGSALVTVIYERSKRPSDIPSRFSGSTSGSTNTLTITGVQADDEAVYFCGGRDSTEAIFGAGTTLTVL

SEQ ID NO: 180 - protein, 102 aa, synthetic construct
ALTQPSSVSANPGETVKIICSGSSSSYGWYQQKSPGSAPVTLIYYNDKRPSDIPSRFSGSKSGSTATLTITGVQVEDEAVYYCGTGDGSIAIFGAGTTLTVL

SEQ ID NO: 181 - protein, 104 aa, synthetic construct
ALTQPSSVSANPGETVKITCSGSSGSYGWFQQKSPGSAPVTVIYNGNNRPSDIPSRFSGSKSGSMGTLTITGVQADDEAVYFCGSYEGSTDTGIFGAGTTLTVL

SEQ ID NO: 182 - protein, 104 aa, synthetic construct; identical to SEQ ID NO: 181

SEQ ID NO: 183 - protein, 104 aa, synthetic construct
ALTQPSSVSANPGETVKITCSGGYSAYAYGWHQQKSPGSALVTVIYERNKRPSDIPSRFSGSTSGSTGTLTITGVQADDEAVYYCGSTDSSTTIFGAGTTLTVL

SEQ ID NO: 184 - protein, 104 aa, synthetic construct; identical to SEQ ID NO: 181

SEQ ID NO: 185 - protein, 104 aa, synthetic construct; identical to SEQ ID NO: 181

SEQ ID NO: 186 - protein, 103 aa, synthetic construct
ALTQPSSVSSNLGGTVEITCSGGGPKSYYGWYQQKSPGSAPVTVIYANDKRPSDIPSRFSGSKSGSTHTLTITGVQAEDEAVYFCGDIDTSGGFGAGTTLTVL

SEQ ID NO: 187 - protein, 104 aa, synthetic construct; identical to SEQ ID NO: 181

SEQ ID NO: 188 - protein, 104 aa, synthetic construct; identical to SEQ ID NO: 181

SEQ ID NO: 189 - protein, 103 aa, synthetic construct
ALTQPSSASANPGETVKITCSGGYSGYAYGWYQQKSPGSALVTVIYERSKRPSDIPSRFSGSTSDSTNTLTITGVQADDEAVYFCGGHDSSSIFGAGTTLTVL

SEQ ID NO: 190 - protein, 103 aa, synthetic construct
ALTQPSSVSANPGETVKITCSGGYSDYAYGWYQQKSPGSALVTVIYERNKRPSNIPSRFSGSRSGSTNTLTITGVQADDEAVYFCGGHDSSSIFGAGTTLTVL

SEQ ID NO: 191 - protein, 103 aa, synthetic construct
ALTQPSSVSANPGETVKITCSGGRYFGWFQQKAPGSAPVTLIYDDTNRPSNIPSRFSGSTSGSTSTLTITGVRAEDEAVYYCGGYDSSTYAGIFGAGTTLTVL

SEQ ID NO: 192 - protein, 104 aa, synthetic construct; identical to SEQ ID NO: 165

SEQ ID NO: 193 - protein, 104 aa, synthetic construct; identical to SEQ ID NO: 165

SEQ ID NO: 194 - protein, 104 aa, synthetic construct; identical to SEQ ID NO: 165

SEQ ID NO: 195 - protein, 107 aa, synthetic construct
ALTQPSSVSANPGETVKITCSGGSSSSYYGWYQQKSPGSAPVTLIYYNDKRPSDIPSRFSGSKSGSTATLTITGVQVEDEAVYYCGSADSSDSGSGIFGAGTTLTVL

SEQ ID NO: 196 - protein, 105 aa, synthetic construct
ALTQPSSVSANPGETVKITCSGGGSSYGWYQQKSPGSAPVTVIYGNDRRPSDIPSRFSGSASGSTATLTITGVQAEDEAVYFCGSADSSDSGSGIFGAGTTLTVL

SEQ ID NO: 197 - protein, 104 aa, synthetic construct; identical to SEQ ID NO: 165

SEQ ID NO: 198 - protein, 104 aa, synthetic construct; identical to SEQ ID NO: 165

SEQ ID NO: 199 - protein, 104 aa, synthetic construct
ALTQPSSVSANLGGTVKITCSGGSSSSYYGWYQQKSPGSAPVTLIYYNDKRPSDIPSRFSGSKSGSTATLTITGVQVEDEAVYYCGTGDGSFAIFGAGTTLTVL

SEQ ID NO: 200 - protein, 104 aa, synthetic construct
ALTQPSSVSANPGETVKITCSGGSGSYGWYQQKTPGSAPVTVIYDNTNRPSNIPSRFSGSTSGSTGTLTITGVQVEDEAVYYCGAYDSSSYIGIFGAGTTLTVL

SEQ ID NO: 201 - skipped

SEQ ID NO: 202 - protein, 8 aa, synthetic construct
YSYYGTYY

SEQ ID NO: 203 - skipped

SEQ ID NO: 204 - protein, 5 aa, synthetic construct
SGSSY

SEQ ID NO: 205 - protein, 5 aa, synthetic construct
SSSSY

SEQ ID NO: 206 - protein, 5 aa, synthetic construct
GSSSY

SEQ ID NO: 207-209 - skipped (3 entries)

SEQ ID NO: 210 - protein, 4 aa, synthetic construct
GSSY

SEQ ID NO: 211-212 - skipped (2 entries)

SEQ ID NO: 213 - protein, 8 aa, synthetic construct
DSWYGSYY

SEQ ID NO: 214 - protein, 5 aa, synthetic construct
GGSSY

SEQ ID NO: 215-216 - skipped (2 entries)

SEQ ID NO: 217 - protein, 5 aa, synthetic construct
GSSSY

SEQ ID NO: 218 - protein, 5 aa, synthetic construct
SSSSY

SEQ ID NO: 219-223 - skipped (5 entries)

SEQ ID NO: 224 - protein, 4 aa, synthetic construct
YSDY

SEQ ID NO: 225 - skipped

SEQ ID NO: 226 - protein, 5 aa, synthetic construct
GPKSY

SEQ ID NO: 227-228 - skipped (2 entries)

SEQ ID NO: 229 - protein, 4 aa, synthetic construct
YSGY

SEQ ID NO: 230 - protein, 4 aa, synthetic construct
YSDY

SEQ ID NO: 231 - skipped

SEQ ID NO: 232 - protein, 5 aa, synthetic construct
SSSSY

SEQ ID NO: 233 - protein, 5 aa, synthetic construct
SSSSY

SEQ ID NO: 234 - protein, 5 aa, synthetic construct
SSSSY

SEQ ID NO: 235 - protein, 5 aa, synthetic construct
SSSSY

SEQ ID NO: 236 - skipped

SEQ ID NO: 237 - protein, 5 aa, synthetic construct
SSSSY

SEQ ID NO: 238 - protein, 5 aa, synthetic construct
SSSSY

SEQ ID NO: 239 - protein, 5 aa, synthetic construct
SSSSY

SEQ ID NO: 240-280 - skipped (41 entries)

SEQ ID NO: 281 - protein, 12 aa, synthetic construct
GSIDSSVGYVGI

SEQ ID NO: 282 - protein, 10 aa, synthetic construct
GSYDSSDSGI

SEQ ID NO: 283 - protein, 10 aa, synthetic construct
GSRDSSYVGL

SEQ ID NO: 284 - protein, 9 aa, synthetic construct
GTGDGSIAI

SEQ ID NO: 285 - protein, 9 aa, synthetic construct
GTGDGSFAI

SEQ ID NO: 286 - protein, 12 aa, synthetic construct
GSADSSDSGSGI

SEQ ID NO: 287 - protein, 13 aa, synthetic construct
GSYDSSAGYAGGI

SEQ ID NO: 288 - protein, 11 aa, synthetic construct
AGYDSSSYVGI

SEQ ID NO: 289 - protein, 8 aa, synthetic construct
GSRDSTTI

SEQ ID NO: 290 - protein, 9 aa, synthetic construct
GTGDGSIAI

SEQ ID NO: 291 - protein, 12 aa, synthetic construct
GSYEGIISFVGI

SEQ ID NO: 292 - protein, 11 aa, synthetic construct
AGYDSSSYVGI

SEQ ID NO: 293 - protein, 11 aa, synthetic construct
GAWDSISDVGI

SEQ ID NO: 294 - protein, 9 aa, synthetic construct
ATGDGTYAV

SEQ ID NO: 295 - protein, 11 aa, synthetic construct
GGYDRSNYNSI

SEQ ID NO: 296 - protein, 10 aa, synthetic construct
GSFDSSVTGI

SEQ ID NO: 297 - protein, 9 aa, synthetic construct
GGYDSSAGI

SEQ ID NO: 298 - protein, 9 aa, synthetic construct
GTGDGSFAI

SEQ ID NO: 299 - protein, 9 aa, synthetic construct
GGRDSTEAI

SEQ ID NO: 300 - protein, 9 aa, synthetic construct
GTGDGSIAI

SEQ ID NO: 301 - protein, 11 aa, synthetic construct
GSYEGSTDTGI

SEQ ID NO: 302 - protein, 11 aa, synthetic construct
GSYEGSTDTGI

SEQ ID NO: 303 - protein, 9 aa, synthetic construct
GSTDSSTTI

SEQ ID NO: 304 - protein, 10 aa, synthetic construct
GGHDSSGSTI

SEQ ID NO: 305 - protein, 11 aa, synthetic construct
GSYEGSTDTGI

SEQ ID NO: 306 - protein, 8 aa, synthetic construct
GDIDTSGG

SEQ ID NO: 307 - protein, 11 aa, synthetic construct
GSYEGSTDTGI

SEQ ID NO: 308 - protein, 11 aa, synthetic construct
GSYEGSTDTGI

SEQ ID NO: 309 - protein, 8 aa, synthetic construct
GGHDSSSI

SEQ ID NO: 310 - protein, 8 aa, synthetic construct
GGHDSSSI

SEQ ID NO: 311 - protein, 11 aa, synthetic construct
GGYDSSTYAGI

SEQ ID NO: 312 - protein, 9 aa, synthetic construct
GTGDGSFAI

SEQ ID NO: 313 - protein, 9 aa, synthetic construct
GTGDGSFAI

SEQ ID NO: 314 - protein, 9 aa, synthetic construct
GTGDGSFAI

SEQ ID NO: 315 - protein, 12 aa, synthetic construct
GSADSSDSGSGI

SEQ ID NO: 316 - protein, 12 aa, synthetic construct
GSADSSDSGSGI

SEQ ID NO: 317 - protein, 9 aa, synthetic construct
GTGDGSFAI

SEQ ID NO: 318 - protein, 9 aa, synthetic construct
GTGDGSFAI

SEQ ID NO: 319 - protein, 9 aa, synthetic construct
GTGDGSFAI

SEQ ID NO: 320 - protein, 11 aa, synthetic construct
GAYDSSSYIGI

SEQ ID NO: 321 - DNA, 43 bp, synthetic construct
  source 1..43: mol_type = other DNA
gtgaccacaggcgtccacagcgccgtgacgttggacgagtccg

SEQ ID NO: 322 - DNA, 46 bp, synthetic construct
  source 1..46: mol_type = other DNA
cacgctaggtcccttggtcgaagcggaggagacgatgacttcggtc

SEQ ID NO: 323 - DNA, 41 bp, synthetic construct
  source 1..41: mol_type = other DNA
ggctgacagacgccaggtgcgcgctgactcagccgtcctcg

SEQ ID NO: 324 - DNA, 41 bp, synthetic construct
  source 1..41: mol_type = other DNA
ggagcggccttaggctggcctaggacggtcagggttgtccc

SEQ ID NO: 325-331 - skipped (7 entries)

SEQ ID NO: 332 - DNA, 44 bp, synthetic construct
  source 1..44: mol_type = other DNA
ctggaattcgtcgcagctatttaatgatggacagcctacgcgac

SEQ ID NO: 333 - DNA, 44 bp, synthetic construct
  source 1..44: mol_type = other DNA
tgtaaaacgacggccagtctattgctacaaatgcctatgcatcc

SEQ ID NO: 334 - DNA, 36 bp, synthetic construct
  source 1..36: mol_type = other DNA
aggaaacagctatgacccaccggttcggggaagtag

SEQ ID NO: 335 - DNA, 18 bp, synthetic construct
  source 1..18: mol_type = other DNA
tgtaaaacgacggccagt

SEQ ID NO: 336 - DNA, 18 bp, synthetic construct
  source 1..18: mol_type = other DNA
caggaaacagctatgacc

SEQ ID NO: 337 - protein, 124 aa, synthetic construct
AVTLDESGGGLQTPGGALSLVCKASGFKFSDHAIFWVRQAPDKGLEFVAAISQGSGSWTAYATVVKGRATISRDNGQSTVRLQLNNLRAADTATYYCAKGAGRACCYAGSIDAWGHGTEVIVSS

SEQ ID NO: 338 - protein, 8 aa, synthetic construct
GFKFSDHA

SEQ ID NO: 339 - protein, 9 aa, synthetic construct
ISQGSGSWT

SEQ ID NO: 340 - protein, 16 aa, synthetic construct
AKGAGRACCYAGSIDA

SEQ ID NO: 341 - protein, 104 aa, synthetic construct; identical to SEQ ID NO: 165

SEQ ID NO: 342 - protein, 4 aa, synthetic construct
SSSY

SEQ ID NO: 343 - skipped

SEQ ID NO: 344 - protein, 9 aa, synthetic construct
GTGDGSFAI